(19) United States
(12) Patent Application Publication
(10) Pub. No.: US 2003/0186826 A1
(43) Pub. Date: Oct. 2, 2003

(54) Method of using personal care compositions containing a high density, water disintegratable, polymeric foam

(75) Inventors: Wayne Ellis Eccard, Cleves, OH (US); Edward Dewey Smith III, Mason, OH (US); John Christopher Wesner, Liberty Township, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/035,380

(22) Filed: Nov. 1, 2001

(51) Int. Cl. A61K 7/50

(52) U.S. Cl. 510/130; 510/119; 510/475

(57) ABSTRACT

Disclosed is a method of topically applying active ingredients to the hair or skin, said method comprising the steps of: (A) applying to the hair or skin a combination of water and a personal care composition, said composition comprising a high-density solid polymeric foam having an Foam Density of from about 0.23 grams/cm³ to about 1.20 grams/cm³, said polymeric foam comprising: (i) a solid polymer structurant; and (ii) an active ingredient comprising a surfactant in a weight ratio of the surfactant to the solid polymer structurant of from about 1:8 to about 10:1; and (B) rinsing the hair or skin with water to disintegrate and rinse-away the high-density polymeric foam such that the disintegrated polymeric foam contains no individual foam pieces having a topographical area of more than about 10% of the original topographical area of the solid polymeric foam prior to application, as determined in accordance with a Substrate Disintegration Test herein. The method provides effective topical delivery of active ingredients from a high-density foam substrate that slowly disintegrates and is rinsed away during prolonged single-use or other similar application.

### FIELD OF THE INVENTION

The present invention relates to a method of using personal care compositions containing a high density, water disintegratable, polymeric foam for topical application to the hair or skin. The high density foam slowly disintegrates when used with water during prolonged application.

### BACKGROUND OF THE INVENTION

Personal care substrates are well known for a variety of applications, including topical application to human skin or other applied surface to provide such surfaces with cleansing, moisturizing, depositing of actives or other materials, disinfecting, or any other effect that can be accomplished by application of a topical substrate to the desired area. These personal care substrates typically comprise a water-insoluble substrate that is either woven or non-woven, and includes products such as disposable baby wipes, sanitizing hand wipes, moisturizing wipes, disposable cleansing wipes, and so forth. These substrates are often single-use products that contain a non-woven or paper substrate that is disposed of immediately after application.

Many of the personal care substrates that are currently known or otherwise available for application of active ingredients to the hair or skin require disposal of a used substrate shortly after application. Although these substrates will eventually dissolve or disperse in water over long periods of time, the extended dissolution is typically designed for disintegration systems such as septic or sewage systems. These prolonged dissolution substrates, however, are typically made of water-insoluble nonwoven materials that do not sufficiently dissolve or disperse during a single-use application, and thus it is still necessary to dispose of used or spent substrates immediately after each application. To avoid the need to dispose of such substrates immediately after use, it is known that these substrates can be manufactured from a solid, water-soluble or dispersible matrix that readily dissolves during application, and thus does not result in used or spent substrates in need of disposal after application.

Water-soluble or dispersible substrates are known in which the base matrix that forms the substrate is a thin sheet or film derived from a solid soap or other surfactant matrix, within which other active ingredients can be trapped or otherwise contained. These water-soluble sheets or films, more commonly referred to as soap sheets, are especially useful as unit dose or single-use cleansing products that readily dissolve when combined with water, but often require the use of a separate cleansing substrate such as a conventional wash cloth or sponge to provide mechanical cleansing action.

Still other personal care compositions have been described in which a cleansing substrate is formed from a water-soluble polymeric matrix that contains a polymeric structurant and a surfactant or other active ingredient. These personal care compositions are then used as cleansing or depositing substrates that provide some mechanical cleansing from the substrate itself, as well as delivery of the active ingredient to the desired site of application. These substrates, however, are typically in the form of stiff, low-density, freeze-dried foams that quickly dissolve when combined with water. Although these stiff foams provide some mechanical cleansing or other substrate benefits to the skin initially, they typically dissolve too rapidly to provide any prolonged cleansing and depositing benefits from a personal care substrate when used with water during prolonged single-use applications such as during a shower or bath.

It has now been found that personal care substrates can be used to apply active ingredients to the hair or skin, wherein the substrate is formulated with water-disintegratable substrates that are soft and flexible prior to and during application, and which provide sustained mechanical cleansing during prolonged single-use application. These personal care substrates slowly dissolve and disintegrate during topical application to the hair or skin, and when the application is complete, the disintegrated substrate rinses away with water, thus obviating the need to dispose of a spent or used substrate. It has also been found that these slowly-disintegrating substrates provide more effective delivery of skin active ingredients than many non-disintegrating substrates in that the active ingredients that are entrapped within the disintegrating substrate are made readily available to the hair or skin during the slow dissolution and disintegration process.

It has been found that such a method of sustained application of active ingredients to the hair or skin can be accomplished by the use of a substrate or composition comprising a solid polymeric foam that contains (A) a solid polymer-containing structurant; and (B) an active ingredient comprising a surfactant at a weight ratio of the surfactant to the polymer-containing structurant of from about 1:8 to about 10:1, wherein the polymeric foam is a high density material as defined by either an Immersion Density or Calculated Density of from about 0.23 grams/cm³ to about 1.20 grams/cm³. The high density foams used in such a method are water-disintegratable substrates that provide sustained topical delivery of surfactants or other active ingredients while only slowly disintegrating and rinsing away with water during prolonged single use or other similar application.

It is therefore an object of the present invention to provide a method of using a personal care composition that provides sustained topical delivery of a surfactant or other active ingredient, wherein the personal care composition comprises a solid polymeric foam. It is a further object of the present invention to provide such a method in that the personal care composition only slowly disintegrates during prolonged single-use application in the presence of water, and which disintegrates and is rinsed away only after such prolonged single-use application. It is a further object of the present invention to provide such a method of using a personal care composition, wherein the composition is in the form of a soft, flexible cleansing substrate.

### SUMMARY OF THE INVENTION

The present invention is directed to a method of topically applying active ingredients from a personal care composition to the hair or skin, wherein the method comprising the steps of: (A) applying to the hair or skin a combination of water and a personal care composition, said personal care composition comprising a high-density solid polymeric foam having an Immersion Density Calculated Density of from about 0.23 grams/cm³ to about 1.20 grams/cm³, said polymeric foam comprising: (i) a solid polymer-containing structurant; and (ii) an active ingredient comprising a surfactant in a weight ratio of the surfactant to the solid polymer-containing structurant of from about 1:8 to about 10:1; and (B) rinsing the hair or skin with water to slowly disintegrate and rinse-away the high-density polymeric foam such that the disintegrated and rinsed-away foam contains no individual foam pieces having a topographical area of more than about 10% of the original topographical area of the solid polymeric foam prior to application, as determined by the Substrate Disintegration Test herein.

The method of the present invention provides sustained topical delivery of active ingredients from a high-density foam substrate that slowly disintegrates and is rinsed away during prolonged single-use or other similar application. It has been found that the polymeric foams used in the methods of the present invention can be formulated as soft, flexible substrates that slowly dissolve or otherwise disintegrate in the presence of water during prolonged single use or other similar application. During such usage, the polymeric foams provide sustained delivery of active ingredients to the desired site of application, while also providing a slowly disintegrating substrate that mechanically delivers the actives to the hair or skin while also partially or completely disintegrating and washing away during application.

It has been found that such a polymeric foam composition for use in the method of the present invention can be prepared by selecting the weight ratio of the surfactant to the solid polymer structurant in the formulation, by selecting the solid polymer structurant with a solubility profile that provides for a slowly disintegrating polymeric foam, and by controlling or maintaining the Foam Density as defined by the ranges described herein.

### DETAILED DESCRIPTION

The personal care compositions for use in the methods of the present invention comprise a solid polymeric foam that contains a solid polymer-containing structurant and an active ingredient including a surfactant. These and other essential limitations of the methods of the present invention, as well as many of the optional ingredients suitable for use herein, are described in detail hereinafter.

The term “solid polymeric foam” as used herein, unless otherwise specified, refers to a solid, interconnected, polymer-containing matrix that defines a network of connected or disconnected, open or closed, cells or spaces that contain a fluid or gas, typically a gas such as air, wherein the cells or spaces generally represent from about 3% to about 80% by volume of the solid polymeric foam.

All viscosity values as described herein, unless otherwise specified, are characterized in centipoises (cP) units and are measured using a TA Instruments AR2000 Rheometer with 4.0 cm diameter parallel plate and 1,000 micron gap at a shear rate of 2.5 reciprocal seconds for a period of 30 seconds at 23° C.

The term “volatile” as used herein, unless otherwise specified, refers to those materials having an average boiling point at one (1) atmosphere of pressure (atm) of less than about 250° C., more typically less than about 235° C. at one (1) atm.

The term “ambient conditions” as used herein, unless otherwise specified, refers to surrounding conditions at one atmosphere of pressure, 50% relative humidity, and 23° C.

All percentages, parts and ratios as used herein are by weight of the total composition, unless otherwise specified.

The personal care method of the present invention can comprise, consist of, or consist essentially of the essential elements and limitations of the invention described herein, as well as any additional or optional ingredients, components, or limitations described herein or otherwise useful in personal care substrates or compositions intended for topical application to the hair or skin.

### Product Form

The personal care compositions for use in the method of the present invention can be formulated in any of a variety of product forms, including solid polymeric foams used alone or in combination with other personal care components. The solid polymeric foams can be continuous or discontinuous when used in the personal care compositions. Regardless of the product form, the key to all of the product form embodiments contemplated within the scope of the method of the present invention is the selected and defined high-density solid polymeric foam that comprises a combination of a solid polymeric structurant and a surfactant-containing active ingredient.

Non-limiting examples of product form embodiments for use in the methods of the present invention include hand cleansing substrates, hair shampoo or other hair treatment substrates, body cleansing substrates, pet care substrates, personal care substrates containing pharmaceutical or other skin care active, moisturizing substrates, sunscreen substrates, chronic skin benefit agent substrates (e.g., vitamin-containing substrates, alpha-hydroxy acid-containing substrates, etc.), deodorizing substrates, fragrance-containing substrates, and so forth.

The product form embodiments for use in the methods of the present invention include substrates having a textured surface, which can be patterned or not, but which have been found to provide enhanced lathering characteristics, increased mechanical exfoliation of the skin, and increased mechanical cleansing of the skin as compared to untextured substrates. The textured substrate preferably results from the shape of the substrate, in that the outermost surface of the substrate contains portions that are raised with respect to other areas of the surface. The raised portions can result from the formed shape of the article, for example the article can be formed originally in a waffle pattern. The raised portions can also be the result of creping processes, imprinted coatings, embossing patterns, laminating to other layers having raised portions, or the result of the physical form of the foam substrate itself, in that it can contain ligaments such as partially collapsed foam cells, e.g., a reticulated foam structure. The texturing can also be the result of laminating the substrate to a second substrate which is textured. The textured substrate is also preferably permeable.

The personal care compositions for use in the methods of the present invention can also be characterized as rinse-off product forms. In this context, the term “rinse-off” means that the solid polymeric foam component of the personal care compositions are applied topically with water to the hair or skin, and then subsequently, but slowly, disintegrated in the presence of water and rinsed away during and immediately after application.

The method of the present invention is not intended for, and specifically excludes, leave-on formulations and applications, e.g., diaper products, bandages, and so forth. The personal care compositions and corresponding methods of application of the present invention also specifically exclude those application methods directed to the application to any surface other than the hair, nails or skin, e.g., excludes methods associated primarily with hard surface cleaners, laundry products, or other similar or related applications. The personal care compositions and corresponding methods of applications of the present invention can be used or otherwise applied topically to humans and other animals such as cats, dogs, horses, livestock, and so forth.

### Solid Polymeric Foam

The solid polymeric foam component of the personal care composition for use in the method of the present invention is a high-density foam that slowly disintegrates when combined with water during single-use application to the hair or skin. The polymeric foam is rinsed away as it slowly disintegrates during application, until the foam is preferably completely disintegrated and washed away during such application and/or a sufficiently small volume of material remains after the desired number of applications, preferably after one application, so that the foam is easy to discard.

The solid polymeric foam component of the personal care compositions for use in the method of the present invention is a solid polymer-containing matrix within which are cells or spaces filled with liquid or gas, most typically a gas such as air. These solid polymeric foams most generally contain from about 2% to about 80% by volume of such cells or spaces, more typically from about 10% to about 70%, by volume of such cells or spaces with respect to the total polymeric foam volume.

### Foam Density

The solid polymeric foam component of the personal care composition for use in the method of the present invention has a selectively high density range as characterized by either an Immersion Density or a Calculated Density, each measured or otherwise determined in accordance with the methodologies as described herein. The Immersion Density is most typically used to characterize substantially closed foam structures, whereas the Calculated Density is most typically used to characterize open cell or reticulated foams that are not as easily measured by the Immersion Density Method. The solid polymeric foam component of the personal care compositions herein includes both types of solid polymeric foam matrices.

As used herein, the term “Foam Density” refers generally to a density measurement or determination as characterized by an Immersion Density, Calculated Density, or both. These high density foam components of the personal care compositions herein have a preferred Immersion Density of from about 0.23 to about 1.2 grams/cm³, more preferably from about 0.25 to about 0.90 grams/cm³, even more preferably from about 0.25 to about 0.80 grams/cm³, or a Calculated Density of from about 0.23 to about 1.20 grams/cm³, preferably from about 0.23 to about 0.80 grams/cm³, even more preferably from about 0.23 to about 0.7 grams/cm³.

The Immersion Density of the solid polymeric foam component (i.e., substrate or piece thereof, designated as substrate segment) is obtained by measuring substrate weight, and the buoyant force of a substrate during immersion in a high viscosity fluid of known density such as honey having a viscosity of from about 15,000 to about 25,000 cP at 25° C. The high viscosity keeps the fluid from rapidly penetrating the pores in the substrate, so that a density comprising primarily the exterior architecture of the substrate segment is obtained. Viscosity of the honey can be adjusted by the water content of the honey, by adding water or drying, as indicated by the National Honey Board, Longmont, Colo., U.S.A. The density of the immersion fluid, ρ_{f}, is obtained by weighing a measured volume in a 250 ml graduated cylinder, and is about 1.424 gm/cm³. About 500 ml of immersion fluid is placed in a clear-walled beaker, for example a glass, 800 ml beaker, and allowed to stand (covered) to de-aerate for 24 hours. A segment of a sample substrate is cut and a balance is used to determine its weight, W₁. The beaker containing immersion fluid is placed on an analytical balance, and the balance is zeroed. The cut substrate segment is immersed in the fluid and then held immobilized, by a thin, rigid immersion wire clamped at the other end to a stand. The cut substrate segment is immersed using the immersion wire to force the substrate segment to immerse at a slow enough rate to keep visible air pockets from forming around the substrate segment as the immersion fluid flows into intimate contact. Upon full immersion, the wire is clamped to the stand in a vertical position with the wire pushing on the center mass of the substrate segment. A timer is started immediately upon clamping the wire to the stand. The substrate segment is checked to ensure it is not touching the sides or bottom of the beaker, nor does its proximity to the surface of the immersion fluid distend the surface of the fluid. The substrate segment remains motionless, which normally occurs when the immersion wire is pushing at the center of mass of the substrate segment and the substrate segment has relaxed. The substrate segment is allowed to relax for several seconds. When the balance reading stabilizes, the buoyant force is recorded as the weight on the balance, W₂. Normally, a constant reading over 4 to 5 seconds is considered stable. If the reading does not stabilize, the buoyant force after 25 seconds immersion is recorded as the buoyant force. The influence of the wire volume is measured by separately taring the beaker on the balance, immersing the wire only to the same depth as during the substrate segment buoyancy measurement, clamping the wire to the stand, and reading the buoyant force due to the wire as the weight on the balance, W₃.

The Immersion Density of the solid polymeric foam (substrate) is then calculated in accordance with the equation: Immersion Density=[W₁/(W₂−W₃)]×ρ_{f}. As determined by Immersion Density Method herein, the Immersion Density of the solid polymeric foam described herein is preferably from about 0.23 to about 1.20 gm/cm³, more preferably from about 0.23 to about 1.0 gm/cm³, more preferably from about 0.25 to about 0.90 gm/cm³, even more preferably from about 0.30 to about 0.85 gm/cm³, and most preferably from about 0.30 to a bout 0.82 gm/cm³.

The solid polymeric foam component of the personal care compositions described herein can also be characterized in terms of a Calculated Density, which is especially useful for those solid polymeric foam embodiments that have an open cell or reticulated foam matrix, and thus are not easily characterized in terms of the Immersion Density Method described hereinbefore.

The Calculated Density of the solid polymeric foam is determined by the equation: Calculated Density=Basis Weight of foam/(Foam Thickness x 1,000), wherein the solid polymeric foam has a preferred Calculated Density of from about 0.23 to about 1.20 gm/cm³, preferably from about 0.23 to about 0.9 g/cm³, more preferably from about 0.25 to about 0.8 g/cm³, most preferably from about 0.25 to about 0.7 g/cm³. The Basis Weight and Foam Thickness of the solid polymeric foams are determined in accordance with the methodologies described hereinafter.

When the solid polymeric foam component of the present invention contains macroscopic pores or holes that describe an open area, for example shaped holes greater than about 1 mm, the sum of the area of these pores or holes on the surface of the foam divided by the total surface area of the foam is designated as the fraction of open area, φa. When such macroscopic holes or pores are present, the Calculated Density is adjusted for their presence by dividing the Calculated Density by the factor, (1−φa).

### Foam Disintegration

The solid polymeric foam component of the personal care compositions for use in the method of the present invention slowly disintegrates in the presence of water during prolonged single-use application. For purposes of defining the polymeric foam component for use in the method herein, the water-dependent disintegration rate of the solid polymeric foam is characterized in accordance with the following Substrate Disintegration Test.

For purposes of defining the personal care compositions used in the method of the present invention, the Substrate Disintegration Test is a measure of the water-disintegration character of a foam substrate, wherein the disintegration must be sufficiently slow to allow for prolonged usage of an intact substrate during topical application with water, but sufficiently rapid or fast to disintegrate and rinse away during the course of such prolonged usage.

In a 400 ml beaker, 200+/−1 grams of distilled water is weighed at room temperature. A 1.0 inch magnetic stir bar is added. Into the water, a 1.0 in² of the sample substrate to be tested is placed. The beaker is placed on a stirring hot plate, for example a Corning model PC-351 Laboratory Hot Plate/Stirrer available from Northwest Scientific Supply Ltd., Victoria, BC, Canada. The stirrer is turned on at a rate sufficient to create a slight vortex in the water, but not so high that the vortex reaches the bottom of the beaker, to prevent possible foaming. The heater is turned on high and the solution heated from room temperature (23° C.) to 90° C. at a rate of about 4.5° C./minute, while continuing to stir. The sample substrate is observed. Stirring is halted momentarily to observe the substrate if needed. After the water reaches 60° C., the top surface and the vortex are stirred with a small spatula in order to separate pockets of accumulated fragments. When the water temperature reaches 90° C., the heat is lowered to moderate and the temperature maintained at between 90° C. and 95° C. for the duration of the test. The sample substrate is observed for a total of 20 minutes, after which the test is stopped. At no time during the test should the magnetic stirrer act as an agitator or beater against the sample substrate.

The solid polymeric foam component of the present invention is water-disintegratable in accordance with the above-described Substrate Disintegration Test, if after the 20 minute test period described above, there are no individual substrate or foam pieces remaining that have a topographical area that represents more than 10% of the original topographical area of the sample substrate or foam tested, and preferably have no foam pieces with a topographical area of greater than 0.10 in².

### Foam Thickness

The solid polymeric foam component of the present invention is preferably a thin, flexible substrate having a Foam Thickness of from about 0.2 mm to about 60 mm, more preferably from about 0.75 mm to about 20 mm, even more preferably from about 1.0 mm to about 15 mm, even more preferably from about 1.25 mm to about 10 mm, most preferably from about 1.5 mm to about 7 mm, as measured by the following methodology.

The Foam Thickness of the solid polymeric foam component (i.e., substrate or sample substrate) is obtained using a Mitutoyo Corporation Digital Disk Stand Micrometer Model Number IDS-1012E (Mitutoyo Corporation, 965 Corporate Blvd, Aurora, Ill., USA 60504) or other similar instrument. The micrometer has a 1 inch diameter platen weighing about 32 grams, which measures thickness at an application pressure of about 40.7 gsi (6.32 gm/cm²).

The thickness of the solid polymeric foam is measured by raising the platen, placing a section of the sample substrate on the stand beneath the platen, carefully lowering the platen to contact the sample substrate, releasing the platen, and measuring the thickness of the sample substrate in millimeters on the digital readout. The sample substrate should be fully extended to all edges of the platen to make sure thickness is measured at the lowest possible surface pressure, except for the case of more rigid substrates which are not flat. For more rigid substrates which are not completely flat, a flat edge of the substrate is measured using only one portion of the platen impinging on the flat portion of the substrate.

### Foam Basis Weight

The solid polymeric foam component of the personal care composition of the present invention has a preferred basis weight of from about 50 grams/m² to about 3,000 grams/m², more preferably from about 100 grams/m² to about 1,500 grams/m², even more preferably from about 150 grams/m² to about 1,200 grams/m², even more preferably from about 200 grams/m² to about 1,100 grams/m², most preferably from about 250 grams/m² to about 1,000 grams/m².

The Basis Weight of the solid polymeric foam component of the personal care composition herein is calculated as the weight of the solid polymeric foam component per area of the selected foam (grams/m²).

### Foam Dry Drape

The solid polymeric foam component for use herein is preferably in the form of thin flexible substrate that has dry drape characteristics as defined herein. As used herein, the Dry Drape value is an indirect measure of the soft, folding character of a solid polymeric foam (i.e., substrate) before it is exposed to water, a character similar to that of a common washcloth.

Dry Drape Values as used herein are determined under ambient conditions. Dry Drape is determined by cutting a precise, 3.5×1 inch strip of substrate using, for example, a JDC Precision Sample Cutter (Thwing-Albert Instrument Company, Philadelphia, Pa., USA). Two strips are used, cutting a substrate strip each in the machine direction and the cross machine direction of manufacture. If the substrate is insufficiently lengthy in any single direction, then two single strips in the same direction are cut and measured to obtain the desired two-sample average. The substrate is held flat on a surface and the length measured to 0.01 mm with a digital caliper (L₀). Two binder clips, each weighing between 2.86 and 2.89 grams, are attached to the substrate, one clip at each end, attaching within 4 mm of the end (e.g., BC-20 OIC Binder Clips made by Officemate International Corporation, Edson, N.J., USA selected to be in target weight range). The sample substrate with clip weights is hung over a 9.5 mm diameter bar. After 10 seconds, the linear end-to-end distance is measured with the digital caliper (L₁ₐ). The substrate is flipped over, again placed on the bar, and the linear end-to-end distance measured in the same way (L_{1b}). The two measurements are averaged to obtain L₁. Dry Drape is then calculated in accordance with the equation; Dry Drape=100×(1−L₁/L₀). The process is then repeated for the other strip and the results averaged. The maximum value for Dry Drape is about 84 because of the width of the bar and the contact between clips at high Dry Drape values.

The Dry Drape Value as used herein in defining the preferred embodiments of the present invention is a two sample average as noted above. The solid polymeric foam component of the present invention has a preferred Dry Drape Value of from 0 to about 84, more preferably from about 5 to about 80, even more preferably from about 10 to about 80, even more preferably from about 20 to about 80, and most preferably from about 25 to about 80.

### Foam Wet Drape

The solid polymeric foam component described herein is preferably in the form of a thin flexible substrate having wet drape characteristics as defined herein. As used herein, the Wet Drape Value is an indirect measure of the soft, folding character of a solid polymeric foam (i.e., substrate) after it is exposed to water, a character similar to that of a common washcloth after exposure to water.

The Wet Drape Value is determined by first cutting a precise 1.0×3.5 inch strip of sample substrate using, for example, a JDC Precision Sample Cutter. Two such strips are cut from the substrate and used herein to obtain an average Wet Drape Value, with one strip cut lengthwise in the machine direction and the other strip lengthwise in the cross machine direction of manufacture. If the substrate is insufficiently lengthy in any single direction, then two single strips in the same direction are cut and measured to obtain the desired two-sample average. The sample substrate is held flat on a surface and the length (L₀) measured to 0.01 mm with a digital caliper. The sample substrate is fully immersed for 10 seconds in a beaker containing 23° C. water, and then removed and patted to remove excess surface water on both sides for 15 seconds, each end is clipped with the same binder clips used for the Dry Drape measurement during the next 15 seconds, and the substrate is draped across a 9.5 mm diameter bar. After 10 seconds (60 seconds from when the substrate was first immersed), the linear end-to-end distance is measured with the digital caliper (L_{w,a}). The substrate is flipped over, again placed on the bar, and the linear end-to-end distance measured again (L_{w,b}). The two measurements are averaged to obtain L_{w}. Wet Drape is calculated according to the equation; Wet Drape=100×(1−L_{w}/L₀). The process is then repeated for the other strip and the results averaged.

The Wet Drape Value as used herein in defining preferred embodiments of the present invention are a two sample average as noted above. The solid polymeric foam component of the present invention preferably has a Wet Drape Value of from 1 to about 84, more preferably from about 5 to about 84, even more preferably from about 20 to about 84, even more preferably from about 30 to about 84, most preferably from about 40 to about 84.

### Foam Lather Volume

The solid polymeric foam component of the present invention provides a lather profile as described hereafter. The personal care composition containing the foam can be a cleansing or non-cleansing composition with differing lather profiles as noted below. The lather profile as described herein is a combination of a Flash Lather Volume and a Total Lather Volume, both of which are determined in accordance with the following Total Lather Volume Test.

Lather volume of a solid polymeric foam (i.e., substrate or sample substrate) is measured using a graduated cylinder and a tumbling apparatus. A 1,000 ml graduated cylinder is chosen which is marked in 10 ml increments and has a height of 14.5 inches at the 1,000 ml mark from the inside of its base (for example, Pyrex No. 2982). Distilled water (100 grams at 23° C.) is added to the graduated cylinder. The cylinder is clamped in a rotating device which clamps the cylinder with an axis of rotation that transects the center of the graduated cylinder. The substrate is prepared by cutting a square of the sample material that weighs 1.00 grams. The substrate is inserted into the graduated cylinder and the cylinder is capped. The cylinder is rotated at a rate of 30 revolutions in 53 seconds, and stopped in a vertical position to complete the first rotation sequence. A timer is set to allow 30 seconds for the lather thus generated to drain. After 30 seconds of such drainage, the first lather volume is measured to the nearest 10 ml mark by recording the lather height in ml up from the base (including any water that has drained to the bottom on top of which the lather is floating). If the top surface of the lather is uneven, the lowest height at which it is possible to see halfway across the graduated cylinder is the first lather volume (ml). If the lather is so coarse that a single or only a few foam cells (“bubbles”) reach across the entire cylinder, the height at which at least 10 foam cells are required to fill the space is the first lather volume, also in ml up from the base. Foam cells larger than one inch in any dimension, no matter where they occur, are designated as unfilled air instead of lather. Foam that collects on the top of the graduated cylinder but does not drain is also incorporated in the measurement if the foam on the top is in its own continuous layer, by adding the ml of foam collected there using a ruler to measure thickness of the layer, to the ml of foam measured up from the base. The maximum foam height is 1,000 ml (even if the total foam height exceeds the 1,000 ml mark on the graduated cylinder). One minute after the first rotation is completed, a second rotation sequence is commenced which is identical in speed and duration to the first rotation sequence. The second lather volume is recorded in the same manner as the first, after the same 30 seconds of drainage time. A third and fourth sequence are completed and the third and fourth lather volume are measured in the same manner, with the same pause between each for drainage and taking the measurement. The lather results after each sequence are added together and the Total Lather Volume determined as the sum of the four measurements, in ml. The Flash Lather Volume is the result after the first rotation sequence only, in ml, i.e., the first lather volume.

The solid polymeric foam component for use herein has a preferred lather profile in which the Total Lather Volume is from about 500 ml to 4,000 ml, more preferably from about 1,300 ml to about 4,000 ml, even more preferably from about 1,500 ml to about 4,000 ml, even more preferably from about 1,750 ml to about 4,000 ml, and most preferably from about 2,000 ml to about 4,000 ml.

The solid polymeric foam component of the present invention, wherein the solid foam component is a depositing substrate rather than a cleansing substrate, has a preferred lather profile in which the Total Lather Volume is from about 400 ml to about 1,000 ml, preferably from about 400 ml to about 900 ml, more preferably from about 400 ml to about 800 ml, and most preferably from about 400 ml to about 700 ml.

The solid polymeric foam component of the present invention, wherein the solid foam component is a cleansing and depositing substrate, has a preferred lather profile in which the Total Lather Volume is from about 1,000 ml to about 4,000 ml, preferably from about 1,500 ml to about 3,500 ml.

### Foam Dissolution Rate

The solid polymeric foam component of the present invention has a Dissolution Rate that allows the solid polymeric foam to slowly disintegrate during prolonged single use or other similar application with water. The Dissolution Rate of the foam component is determined in accordance with the methodology described below.

Immediately at the end of the Total Lather Volume Test as described hereinbefore, the recoverable substrate is captured by carefully pouring the contents of the graduated cylinder through a cellulose paper towel (e.g., Bounty Towel). All visible substrate pieces that measure ⅛ inch (largest dimension) or larger are gathered and placed on a pre-weighed dish, for example a Falcon Optilux™ Petri Dish (Becton Dickinson and Company, Franklin Lakes, N.J., USA, 07417). The pieces are removed by hand, using no utensils, to avoid capturing semi-dissolved gel material. An additional 100 to 200 ml of cold tap water is added to the graduated cylinder, followed by a second, and if necessary, a third addition of the same amount of water poured through the paper towel to ensure all pieces have been removed from the graduated cylinder. The dish with wet pieces is placed in a still air oven at 160° F. overnight to dry. After 15-18 hours drying, the dish is removed and the dry weight of the recovered substrate pieces, W_{f}, is determined using an analytical balance. Separately, a second piece of the same substrate is weighed (initial weight) and then dried under the same oven conditions and weighed (dry weight) to establish the substrate solids level, W₀. The Dissolution Rate is then calculated using the equations W₀=Dry weight of the second substrate piece÷Initial weight of second substrate piece, and Dissolution Rate=100×(1−W_{f}/W₀).

The solid polymeric foam component of the present invention has a preferred Dissolution Rate of from about 10% to about 100%, more preferably from about 14% to about 100%, even more preferably from about 20% to about 96%, even more preferably from about 25% to about 90%, most preferably from about 27% to about 80%.

### Moisture Retention Methodology

The polymeric foam component of the present invention is preferably substantially dry in that it has a dry feeling prior to use. In this context, the term “substantially dry” means that the polymeric foam has a preferred Moisture Retention value of less than about 0.95 grams, preferably less than about 0.75 grams, even more preferably, less than about 0.5 grams, even more preferably less than about 0.1 grams.

The Moisture Retention value as used to characterize the above-described embodiments of the present invention is an indirect measure of the dry feel of the substrate prior to use, and is determined in accordance with the following methodology.

Two paper towel sheets (Bounty White Paper Towel—SKU 37000 63037, The Procter & Gamble Company, Cincinnati, Ohio USA) are weighed separately. One sheet is placed on a flat surface. A sample substrate is positioned on the sheet, and the second paper towel sheet then placed on top of the positioned substrate. A piece of Lexan plastic large enough to completely cover the sample and weighing 1,000 grams is placed on top of the second towel, and a 2,000 gram weight is placed on top of the Lexan. After one minute, the Lexan and weight are removed and the two paper towels are separated from the sample substrate and weighed.

The Moisture Retention value of the sample substrate is determined by subtracting the initial weight of both paper towels together from the final weight of both towels together after the one minute period described above. The Moisture Retention value for purpose of defining the above-described embodiments is a sample average taken from two or more sample substrate tests.

### Active Ingredient

The solid polymeric foam component of the present invention comprises an active ingredient that includes a surfactant suitable for application to the hair or skin. Suitable surfactants for use herein include any known or otherwise effective surfactant suitable for application to the hair or skin, and which is otherwise compatible with the other essential ingredients in the compositions. The surfactants as an active ingredient for use herein include emulsifying agents as well as cleansing surfactants.

For purposes of defining the personal care compositions for use in the method of the present invention, the active ingredient component shall be any solid or liquid material contained within the solid polymeric foam that is not a polymeric structurant, but shall not include any material that is associated with or fixed to the solid polymeric foam but not contained within the foam, which shall instead be referred to herein as an optional rather than active ingredient. Such associated or fixed optional materials might include, for example, an insoluble woven substrate on which the solid polymer foam is attached but is not otherwise contained within. Other associated or fixed materials might include active ingredients coated onto the foam substrate, but not contained within the foam, and therefore would be considered an optional rather than an active ingredient for invention definition purposes herein.

The active ingredient is a solid or liquid material formulated within the composition such that the weight ratio of the surfactant component of the active ingredient to the solid polymer structurant as described herein ranges from about 1:8 to about 10:1, preferably from about 1:5 to about 3:1, more preferably from about 1:1.6 to about 2:1, even more preferably from about 1:2 to about 2:1. The concentration of the active ingredient within the composition can vary depending variables such as the selected concentration of the solid polymer structurant, the intended product form and use of the composition, the type of active ingredient selected, and so forth.

The active ingredient component of the personal care compositions must include at least a minimal amount of surfactant, although all of the active ingredient can be surfactant, but can also include in addition to the surfactant any other active ingredient that provides a topical benefit when applied to the hair or skin, non-limiting examples of such other ingredients include moisturizers, emollients, pharmaceutical actives, vitamins, nutrients, sunscreens, perfumes or fragrances, deodorants, sensates, astringents, cosmetic particles, absorbent particles, adhesive particles, polymeric particles or films, fibers, dyes, reactive agents, skin lightening agents, skin tanning agents, exfoliating agents, acids, bases, humectants, enzymes, or any other active ingredient that provides a functional cosmetic, aesthetic or other skin active benefit known for or otherwise useful in personal care compositions.

Surfactant concentrations in the solid polymeric foam component of the present invention will vary widely depending upon factors such as the type of product form selected, e.g. lathering or non-lathering, cleansing or non-cleansing, the type of surfactant and other ingredients selected, and so forth. For cleansing or lathering embodiments of the present invention, the surfactant concentration generally ranges from about 0.5% to about 75%, preferably from about 1% to about 70%, more preferably from about 5% to about 65%, most preferably from about 10% to about 60%, by weight of the solid polymeric foam, whereas for non-cleansing or non-lathering embodiments of the present invention, the surfactant concentration generally ranges from about 0.1% to about 60%, more preferably from about 0.5% to about 55%, more preferably from about 1.0% to about 55%, most preferably from about 1.0% to about 50%, by weight of the solid polymeric foam.

Surfactants suitable for use in the solid polymeric foam component of the present invention include anionic surfactants, nonionic surfactants, cationic surfactants, zwitterionic surfactants, amphoteric surfactants, or combinations thereof. The surfactant component of the polymeric foam is essential in preparing a stable structure for the solid polymeric foam component described herein, although it is understood that the surfactant component can also be used to provide in addition to a stable foam structure, a cleansing material for use in the method of the present invention. Likewise, the surfactant component can also be used solely or primarily as a process aid in making a stable foam, wherein the surfactant includes conventional surfactants or emulsifiers that need not provide any lathering performance. Examples of emulsifiers for use as a surfactant component herein include mono- and di-glycerides, fatty alcohols, polyglycerol esters, propylene glycol esters, sorbitan esters and other emulsifiers known or otherwise commonly used to stabilized air interfaces, as for example those used during preparation of aerated foodstuffs such as cakes and other baked goods and confectionary products, or the stabilization of cosmetics such as hair mousses.

Anionic surfactants suitable for use in the personal care compositions of the present invention include those described in McCutcheion's Detergents and Emulsifiers, North American Edition (1986), Allured Publishing Corp.; McCutcheion's, Functional Materials, North American Edition (1992), Allured Publishing Corp.; and U.S. Pat. No. 3,929,678 (Laughlin et al.), which descriptions are incorporated herein by reference.

Non-limiting examples of anionic surfactants suitable for use herein include alkyl and alkyl ether sulfates, sulfated monoglycerides, sulfonated olefins, alkyl aryl sulfonates, primary or secondary alkane sulfonates, alkyl sulfosuccinates, acyl taurates, acyl isethionates, alkyl glycerylether sulfonate, sulfonated methyl esters, sulfonated fatty acids, alkyl phosphates, acyl glutamates, acyl sarcosinates, alkyl sulfoacetates, acylated peptides, alkyl ether carboxylates, acyl lactylates, anionic fluorosurfactants, sodium lauroyl glutamate, and combinations therof.

Anionic surfactants suitable for use in the personal care compositions of the present invention include alkyl and alkyl ether sulfates. These materials have the respective formulae ROSO₃M and RO(C₂H₄O)ₓSO₃M, wherein R is alkyl or alkenyl of from about 8 to about 24 carbon atoms, x is 1 to 10, and M is a water-soluble cation such as ammonium, sodium, potassium and triethanolamine. The alkyl ether sulfates are typically made as condensation products of ethylene oxide and monohydric alcohol's having from about 8 to about 24 carbon atoms. Preferably, R has from about 10 to about 18 carbon atoms in both the alkyl and alkyl ether sulfates. The alcohol's can be derived from fats, e.g., coconut oil or tallow, or can be synthetic. Lauryl alcohol and straight chain alcohol's derived from coconut oil are preferred herein. Such alcohol's are reacted with about 1 to about 10, preferably from about 3 to about 5, and especially about 3, molar proportions of ethylene oxide and the resulting mixture of molecular species having, for example, an average of 3 moles of ethylene oxide per mole of alcohol, is sulfated and neutralized.

Specific examples of alkyl ether sulfates which may be used in the personal care compositions are sodium and ammonium salts of coconut alkyl triethylene glycol ether sulfate; tallow alkyl triethylene glycol ether sulfate, and tallow alkyl hexaoxyethylene sulfate. Highly preferred alkyl ether sulfates are those comprising a mixture of individual compounds, said mixture having an average alkyl chain length of from about 10 to about 16 carbon atoms and an average degree of ethoxylation of from about 1 to about 4 moles of ethylene oxide.

Other suitable anionic surfactants include water-soluble salts of the organic, sulfuric acid reaction products of the general formula [R¹—SO₃-M], wherein R¹ is chosen from the group consisting of a straight or branched chain, saturated aliphatic hydrocarbon radical having from about 8 to about 24, preferably about 10 to about 18, carbon atoms; and M is a cation. Important examples are the salts of an organic sulfuric acid reaction product of a hydrocarbon of the methane series, including iso-, neo-, ineso-, and n-paraffins, having about 8 to about 24 carbon atoms, preferably about 10 to about 18 carbon atoms and a sulfonating agent, e.g., SO₃, H₂SO₄, oleum, obtained according to known sulfonation methods, including bleaching and hydrolysis. Preferred are alkali metal and ammonium sulfonated C₁₀₁₈ n-paraffins.

Additional examples of suitable anionic surfactants are the reaction products of fatty acids esterified with isethionic acid and neutralized with sodium hydroxide where, for example, the fatty acids are derived from coconut oil; sodium or potassium salts of fatty acid amides of methyl tauride in which the fatty acids, for example, are derived from coconut oil. Other suitable anionic surfactants of this variety are described in U.S. Pat. No. 2,486,921, U.S. Pat. No. 2,486,922 and U.S. Pat. No. 2,396,278.

Still other suitable anionic surfactants are the succinamates, examples of which include disodium N-octadecylsulfosuccinamate; diammoniumlauryl sulfosuccinamate; tetrasodium N-(1,2-dicarboxyethyl)-N-octadecylsulfosuccinamate; diamyl ester of sodium sulfosuccinic acid; dihexyl ester of sodium sulfosuccinic acid; and dioctyl esters of sodium sulfosuccinic acid.

Other suitable anionic surfactants include olefin sulfonates having about 12 to about 24 carbon atoms. The term “olefin sulfonates” is used herein to mean compounds which can be produced by the sulfonation of a-olefins by means of uncomplexed sulfur trioxide, followed by neutralization of the acid reaction mixture in conditions such that any sulfones which have been formed in the reaction are hydrolyzed to give the corresponding hydroxy-alkanesulfonates. The sulfur trioxide can be liquid or gaseous, and is usually, but not necessarily, diluted by inert diluents, for example by liquid SO₂, chlorinated hydro-carbons, etc., when used in the liquid form, or by air, nitrogen, gaseous SO₂, etc., when used in the gaseous form.

The a-olefins from which the olefin sulfonates are derived are mono-olefins having about 12 to about 24 carbon atoms, preferably about 14 to about 16 carbon atoms. Preferably, they are straight chain olefins.

In addition to the true alkene sulfonates and a proportion of hydroxy-alkanesulfonates, the olefin sulfonates can contain minor amounts of other materials, such as alkene disulfonates depending upon the reaction conditions, proportion of reactants, the nature of the starting olefins and impurities in the olefin stock and side reactions during the sulfonation process.

Another class of anionic surfactants suitable for use in the personal care compositions are the b-alkyloxy alkane sulfonates. These compounds have the following formula:

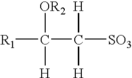

where R₁ is a straight chain alkyl group having from about 6 to about 20 carbon atoms, R₂ is a lower alkyl group having from about 1 (preferred) to about 3 carbon atoms, and M is a water-soluble cation as hereinbefore described.

Other suitable surfactants are described in McCutcheon's, Emulsifiers and Detergents, 1989 Annual, published by M. C. Publishing Co., and in U.S. Pat. No. 3,929,678, which descriptions are incorporated herein by reference.

Preferred anionic surfactants for use in the personal cared compositions include ammonium lauryl sulfate, ammonium laureth sulfate, triethylamine lauryl sulfate, tri-ethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, and combinations thereof.

Amphoteric surfactants suitable for use in the personal care compositions of the present invention includes those that are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition are sodium 3-dodecyl-aminopropionate, sodium 3-dodecylaminopropane sulfonate, sodium lauryl sarcosinate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and the products described in U.S. Pat. No. 2,528,378.

Cationic surfactants can also be used in the personal care compositions of the present invention, but are generally less preferred, and preferably represent less than about 5% by weight of the compositions.

Suitable nonionic surfactants for use as lathering surfactants in the personal care compositions of the present invention include those described in McCutcheion's Detergents and Emulsifiers, North American edition (1986), Allured Publishing Corp., and McCutcheion's Functional Materials, North American edition (1992), which descriptions are incorporated herein by reference. These nonionic lathering surfactants suitable for use herein include alkyl glucosides, alkyl polyglucosides, polyhydroxy fatty acid amides, alkoxylated fatty acid esters, sucrose esters, amine oxides, and combinations thereof.

Suitable nonionic surfactants for use in the personal care compositions of the present invention include condensation products of alkylene oxide groups (hydrophilic in nature) with an organic hydrophobic compound, which may be aliphatic or alkyl aromatic in nature. Preferred classes of nonionic surfactants include:

1) polyethylene oxide condensates of alkyl phenols, e.g., the condensation products of alkyl phenols having an alkyl group containing from about 6 to about 20 carbon atoms in either a straight chain or branched chain configuration, with ethylene oxide, the ethylene oxide being present in amounts equal to from about 10 to about 60 moles of ethylene oxide per mole of alkyl phenol;

2) nonionic surfactants derived from the condensation of ethylene oxide with the product resulting from the reaction of propylene oxide and ethylene diamine products;

3) condensation products of aliphatic alcohol's having from about 8 to about 18 carbon atoms, in either straight chain or branched chain configuration, with ethylene oxide, e.g., a coconut alcohol ethylene oxide condensate having from about 10 to about 30 moles of ethylene oxide per mole of coconut alcohol, the coconut alcohol fraction having from about 10 to about 14 carbon atoms;

4) long chain tertiary amine oxides corresponding to the following general formula:

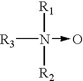

wherein R₁ contains an alkyl, alkenyl or monohydroxy alkyl radical of from about 8 to about 18 carbon atoms, from 0 to about 10 ethylene oxide moieties, and from 0 to about 1 glyceryl moiety, and R₂ and R₃ contain from about 1 to about 3 carbon atoms and from 0 to about 1 hydroxy group, e.g., methyl, ethyl, propyl, hydroxyethyl, or hydroxypropyl radicals;

5) long chain tertiary phosphine oxides corresponding to the following general formula:

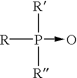

wherein R contains an alkyl, alkenyl or monohydroxyalkyl radical ranging from about 8 to about 18 carbon atoms in chain length, from 0 to about 10 ethylene oxide moieties and from 0 to about 1 glyceryl moiety and R′ and R″ are each alkyl or monohydroxyalkyl groups containing from about 1 to about 3 carbon atoms;

6) long chain dialkyl sulfoxides containing one short chain alkyl or hydroxy alkyl radical of from about 1 to about 3 carbon atoms (usually methyl) and one long hydrophobic chain which include alkyl, alkenyl, hydroxy alkyl, or keto alkyl radicals containing from about 8 to about 20 carbon atoms, from 0 to about 10 ethylene oxide moieties and from 0 to about 1 glyceryl moiety;

7) alkyl polysaccharide (APS) surfactants such as the alkyl polyglycosides, as described in U.S. Pat. No. 4,565,647, which have a hydrophobic group with about 6 to about 30 carbon atoms and polysaccharide (e.g., polyglycoside) as the hydrophilic group, and optionally have a polyalkylene-oxide group joining the hydrophobic and hydrophilic moieties, wherein the alkyl group (i.e., the hydrophobic moiety) can be saturated or unsaturated, branched or unbranched, and unsubstituted or substituted (e.g., with hydroxy or cyclic rings); and

8) polyethylene glycol (PEG) glyceryl fatty esters, such as those of the formula R(O)OCH₂CH(OH)CH₂(OCH₂CH₂)ₙOH wherein n is from about 5 to about 200, preferably from about 20 to about 100, and R is an aliphatic hydrocarbyl having from about 8 to about 20 carbon atoms.

Zwitterionic surfactants suitable for use in the personal care compositions of the present invention include those that are broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. Such suitable zwitterionic surfactants can be represented by the formula:

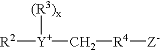

wherein R² contains an alkyl, alkenyl, or hydroxy alkyl radical of from about 8 to about 18 carbon atoms, from 0 to about 10 ethylene oxide moieties and from 0 to about 1 glyceryl moiety; Y is selected from the group consisting of nitrogen, phosphorus, and sulfur atoms; R³ is an alkyl or monohydroxyalkyl group containing about 1 to about 3 carbon atoms; X is 1 when Y is a sulfur atom, and 2 when Y is a nitrogen or phosphorus atom; R⁴ is an alkylene or hydroxyalkylene of from about 1 to about 4 carbon atoms and Z is a radical selected from the group consisting of carboxylate, sulfonate, sulfate, phosphonate, and phosphate groups.

Other zwitterionic surfactants suitable for use herein include betaines, including high alkyl betaines such as coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, and lauryl bis-(2-hydroxypropyl)alpha-carboxyethyl betaine. The sulfobetaines may be represented by coco dimethyl sulfopropyl betaine, stearyl dimethyl sulfopropyl betaine, lauryl dimethyl sulfoethyl betaine, lauryl bis-(2-hydroxyethyl) sulfopropyl betaine and the like; amidobetaines and amidosulfobetaines, wherein the RCONH(CH₂)₃ radical is attached to the nitrogen atom of the betaine are also useful in this invention.

### Structurant

The solid polymeric foam component of the present invention comprises a polymer structurant in solid form or that otherwise forms a solid polymeric matrix within the compositions during or after formulation. Any polymer suitable for use in personal care applications can be used as a structurant herein, so long as it helps provide the polymeric foam with the requisite structure and physical/chemical characteristics as described herein.

The solid polymers selected for use as structurants herein are preferably those polymers or polymer materials that are soluble in hot water but insoluble in cold water. These selected and preferred polymers or polymer materials are those having the requisite cold water insolubility as characterized by a viscosity of less than about 15 cP as determined by the Cold Water Insolubility Test as described herein. These same polymers and polymer materials are also hot water soluble, which means that they have a viscosity greater than about 10 cP as determined in accordance with the Hot Water Solubility Test as described herein.

The solid polymer structurants for use herein can also be characterized in terms of a water solubility profile that selects only those solid polymer structurants having a first viscosity of less than about 15 cP in accordance with the Cold Water Insolubility Test and a second viscosity of greater than about 15 cP in accordance with a Second Cold Water Insolubility Test, all of which are described in detail hereinafter.

The preferred viscosity and water solubility characteristics described above characterize the solid polymers and polymer materials for use as structurants herein. In this context, the term “polymer material” refers to those polymers that when used in combination with other materials results in a polymer-containing structurant that now has above-described water solubility and viscosity characteristics. The polymer materials can therefore include polymers that would not otherwise meet the above-described viscosity and solubility characteristics, but-for their use in combination with other materials. Such other materials are most typically liquid organic solvents or cosolvents, especially water miscible solvents such as isopropanol, ethanol, and other low molecular weight volatile alcohols, physical or chemical cross-linking agents such as formaldehyde or polyvalent ionic metal salts such as magnesium chloride, or precipitants such as pH adjusting acids and bases and coacervate forming agents such as some surfactants, but can also include other materials that will otherwise render a polymeric material useful as a structurant as defined herein.

The solid polymers suitable for use herein can be used alone or in combination with other structurants, polymeric or non-polymeric. These solid polymers can include homopolymers and copolymers, including block, graft, star, and cross-linked polymers, as well as combinations thereof, all of which preferably have an average molecular weight of from about 10,000 to about 2 million, more preferably from about 20,000 to about 1 million, even more preferably from about 70,000 to about 800,000.

The solid polymers as structurants herein include many synthethic as well as natural polymers, including silicone-containing and non-silicone containing polymers, but are preferably synthentic polymers. Non-limiting examples of such solid polymers include polyvinyl alcohols, polyvinyl pyrrolidone, polyalkylene oxides, celluloses and cellulose derivatives such as cellulose ethers and alkhydroxyl celluloses, polysaccharides, and polysaccharide derivatives such as starch acrylate copolymers, polycarboxylic acids and salts such as polyacrylates, polyalkylacrylates, polyamino acids or peptides, polyamides, polyacrylamides, polyesters, poly(vinyl methyl ether-co-maleic anhydride) or derivatives or copolymers thereof. Most preferred are polyvinyl alcohols. The solid polymers as structurants are preferably synthetic polymers.

Other solid polymers suitable for use herein include alginates including alginate derivatives such as propylene glycol alginate, pectins including both low and high degree of methoxylation, polyethylene oxides, gelatins, carrageenans, chitosans, starches and starch derivatives, and combinations thereof.

### Cold Water Insolubility

The solid polymeric structurant for use herein has a preferred water solubility profile as characterized by a Hot Water Solubility Test and a Cold Water Insolubility Test. The solubility profile can alternatively be characterized in terms of the same Cold Water Insolubility Test but now in combination with a Second Cold Water Insolubility Test. The Cold Water Insolubility Test and the Second Cold Water Insolubility Test provide a measure of the cold water insolubility of the solid polymeric structurants for use herein, and is characterized as a viscosity measurement of less than about 15 cP in accordance with the Cold Water Insolubility Test, and a viscosity of more than about 15 cP for the Second Cold Water Insolubility Test, both as described hereinafter.

A) Cold Water Insolubility Test

In a 4-ounce jar, a rheology sample is prepared by blending 71.25 grams distilled water at 23° C. with 3.75 grams dry polymer and capping the jar. Shake vigorously by hand for one minute. Place in an oscillating shaker such as a Lab-Line Shaker Bath by Lab-Line Instruments (Model #3540), Melrose, Ill., USA 60160, and shake for 24 hours at 125-150 oscillations per minute and room temperature. Periodically, remove and vigorously shake by hand. After 24 hours, remove from shaker and measure the viscosity at 2.5 inverse seconds shear rate and 23° C. using, for example, a TA Instruments AR2000 rheometer with a 4 cm diameter parallel plate and 1,000 micron gap. If samples appear non-homogeneous, the rheology sample is drawn from the clearest portion of the sample, i.e., the supernatant. The sample can also be lightly centrifuged in a laboratory centrifuge at a few thousand r.p.m. to clarify a dispersion. The viscosity is reported as the result of the Cold Water Insolubility Test, and is reported in centipoises (cP). Cold water soluble polymers have a viscosity greater than about 15 cP, preferably greater than about 50 cP, and most preferably greater than about 75 cP. Lambda Carrageenan, PEG 14M, xanthan gum, methylcellulose and sodium alginate are cold water soluble and not useful by themselves as the film forming polymer for the invention.

When cosolvents, cosolublizing agents, insolubilizing agents, drying agents, or other agents that affect solubility of the polymer or the polymer film are used in the composition, they can render the polymer or polymer film either more or less soluble or more or less insoluble in cold water. For example, a water soluble polymer can be rendered insoluble or its cold water solubility reduced by physical or chemical crosslinking; or by precipitating as a result of insolubilizing agents such as acids or bases or salts, especially polyvalent ionic salts; or the like. Also for example, the polymer film can be prepared from a mixture of water and other solvents wherein the film is insoluble in water after it is formed. Also for example, the polymer film can be prepared from an emulsion polymer or a mixture of an emulsion polymer and other polymers, wherein the film that is formed is insoluble in cold water. A film forming polymer that is rendered cold water insoluble, for the sake of this invention, is also considered a cold water insoluble polymer. Polymers films are tested in the same manner as the Cold Water Insolubility Test with the following changes. A polymer film is prepared. 75 grams of a sample mixture is prepared by blending 3.75 grams dry polymer or polymers with 71.25 grams water and/or other solvents. Other additives such as solvents, cosolvents, cosolublizing agents, drying agents or insolubilizing agents are added in the same proportion to the polymer as present in the composition, reducing the water/solvent by the same amount. The sample mixture is prepared by agitating as described using the Lab-Line Shaker Bath. If the polymer or polymer blend does not subsequently form a film from this mixture (by casting and drying, e.g., or in a polyethylene dish), then increased agitation and/or heating of the sample mixture is used as necessary to allow the polymer to form a film by casting into a dish and drying. The sample mixture is poured into a polyethylene tray, for example a 14 cm×14 cm high density polyethylene tray, and dried in a still air oven at between 100-160° F. for about 2-3 days, until a film forms and the weight is unchanging, indicating complete solvent drying. The film is then used as the dry polymer in the Cold Water Insolubility Test.

B) Second Cold Water Insolubility Test

In a 4-ounce jar, a rheology sample is prepared by blending 71.25 grams distilled water at 23° C. with 3.75 grams dry polymer and capping the jar. Shake vigorously by hand for one minute. Place in an oscillating shaker filled with water, such as a Lab-Line Shaker Bath by Lab-Line Instruments (Model #3540), Melrose, Ill., USA 60160, and shake for 24 hours at 125-150 oscillations per minute and 80° C. Periodically, remove and vigorously shake by hand. After 24 hours, remove from shaker and measure the viscosity at 2.5 inverse seconds shear rate and a temperature of 23° C. using, for example, a TA Instruments AR2000 rheometer with a 4 cm diameter parallel plate and 1,000 micron gap. If samples appear non-homogeneous, the rheology sample is drawn from the clearest portion of the sample and can be centrifuged lightly as described in the Cold Water Insolubility Test. The viscosity is reported as the result of the Second Cold Water Insolubility Test, and is reported in centipoises (cP).

It has been found that polymers having a Second Cold Water Insolubility Test viscosity greater than about 15 cP are useful polymeric structurants in the compositions of the present invention in that the process of heating the polymers during the Second Cold Water Insolubility Test renders them soluble and thus enables them to be used as polymeric structurants herein, while also maintaining a moderate dissolution rate profile for the dry foam compositions.

C) Hot Water Solubility Test

The solid polymeric structurant for use herein has a preferred water solubility profile as characterized by a Hot Water Solubility Test and a Cold Water Insolubility Test. The Hot Water Solubility Test provides a measure of the hot water solubility of the solid polymeric structurants for use herein, and is characterized as a viscosity measurement greater than about 10 cP, more preferably greater than 20 cP, even more preferably greater than 40 cP, all determined in accordance with the Hot Water Solubility Test as described hereinafter.

In a 4-ounce jar, a rheology sample is prepared by blending 71.25 grams distilled water at 23° C. with 3.75 grams dry polymer and capping the jar. Shake vigorously by hand for one minute. Place in an oscillating shaker filled with water, such as a Lab-Line Shaker Bath by Lab-Line Instruments (Model #3540), Melrose, Ill., USA 60160, and shake for 24 hours at 125-150 oscillations per minute and 80° C. Periodically, remove and vigorously shake by hand. After 24 hours, remove from shaker and measure the viscosity at 2.5 inverse seconds shear rate and a temperature of 80° C. for 15-30 seconds using, for example, a TA Instruments AR2000 rheometer with a 4 cm diameter parallel plate and 1,000 micron gap. If samples appear non-homogeneous, the rheology sample is drawn from the clearest portion of the sample and can be centrifuged lightly as described in the Cold Water Insolubility Test. The viscosity at 80° C. is reported in centipoises (cP) as a result of the Hot Water Solubility Test.

### Optional Ingredients

The personal care compositions of the present invention may further comprise other optional ingredients that are known for use or otherwise useful effective in personal care compositions, provided that such optional materials are compatible with the selected essential materials described herein, or do not otherwise unduly impair product performance.

Such optional ingredients are most typically those materials approved for use in cosmetics and that are described in reference books such as the CTFA Cosmetic Ingredient Handbook, Second Edition, The Cosmetic, Toiletries, and Fragrance Association, Inc. 1988, 1992. Non limiting examples of such optional ingredients include preservatives, perfumes or fragrances, coloring agents or dyes, thickeners, moisturizers, emollients, pharmaceutical actives, vitamins or nutrients, sunscreens, deodorants, sensates, astringents, cosmetic particles, absorbent particles, adhesive particles, fibers, reactive agents, skin lightening agents, skin tanning agents, exfoliating agents, acids, bases, humectants, enzymes, suspending agents, pH modifiers, co-solvents or other additional solvents, and similar other materials.

Preferred optional ingredients include any plasticizing agent suitable for use in personal care compositions. Non-limiting examples of suitable plasticizing agents include glycerol, propylene glycol, butylenes glycol, cyclohexane dimethanol and other mono- and polyhydric low molecular weight alcohols (e.g., C2-C8 alcohols), alkyl and allyl phthallates, napthalates and other low molecular weight esters (e.g., esters of C2-C10 alcohols and acids). These optional plasticizing agents are preferably used as an active ingredient in the solid polymeric foam at a concentration of from about 0.1% to about 30%, more preferably from about 0.5% to about 25%, even more preferably from about 1% to about 20%, most preferably from about 2% to about 15%, by weight of the solid polymeric foam.

Other preferred optional ingredients include organic solvents, especially water miscible solvents and co-solvents useful as solublizing agents for polymeric structurants and as drying accelerators. Non-limiting examples of suitable solvents include alcohols, esters, ketones, aromatic hydrocarbons, aliphatic hydrocarbons, ethers, and combinations thereof. Alcohols and esters are more preferred. Preferred alcohols are monohydric. The most preferred monohydric alcohols are ethanol, iso-propanol, and n-propanol. The most preferred esters are ethyl acetate and butyl acetate. Other non-limiting examples of suitable organic solvents are benzyl alcohol, amyl acetate, propyl acetate, acetone, heptane, iso-butyl acetate, iso-propyl acetate, toluene, methyl acetate, iso-butanol, n-amyl alcohol, n-butyl alcohol, hexane, and methyl ethyl ketone, methanol, ethanol, n-propanol, isopropanol, n-butanol, isobutanol, methylethylketone, acetone, and combinations thereof.

Other preferred optional ingredients include other film forming polymers including cold water soluble film forming polymers, latex or emulsion polymers, thickeners such as water soluble polymers, clays, silicas, ethylene glycol distearate, deposition aids, including coacervate forming components and quaternary amine compounds.

For purposes of defining the personal cleansing care compositions of the present invention, the optional ingredients are considered an active ingredient component when contained within the solid polymeric foam, and as mere optional ingredients when not contained within the polymeric foam.

### Method of Use

The method of the present invention is directed to the topical application of the personal care composition as described herein, wherein the composition is applied to the hair or skin in combination with water. The composition, which is preferably in the form of a cleansing or non-cleansing substrate, is used in a conventional manner on the applied surface to cleanse or otherwise deposit the desired active ingredient to the applied area. In this manner, the substrate is rubbed over or otherwise moved across the applied surface repeatedly during the desired usage period, typically during a shower or bath lasting typically from about 0.5 minutes to about 20 minutes, more typically from about 2 minutes to about 15 minutes, until the solid polymeric foam within the substrate is preferably completely disintegrated and washed or rinsed away.

The method of the present invention is also directed to the above-described method, wherein the substrate is used for more multiple uses or applications, preferably more than a single use, even more preferably from about 1 to about 7 uses, prior to discarding the remaining substrate, if any, down the drain or in a trash receptacle.

The method of the present invention is also directed to the above-described method, wherein the personal care composition is used in either cold, warm or hot water, preferably warm or hot water at a temperature of at least about 85° F., more preferably at least about 95° F., even more preferably from about 100° F. to about 140° F. It has been found that the solid polymeric foam as described herein provides for prolonged dissolution and disintegration even when used in combination with the warm or hot water. Thus, these personal care compositions as described herein are especially effective as cleansing substrates when used in warm/hot showers or baths as an alternative to other more traditional cleansing substrates such as washcloths with added soaps or other cleansing surfactant compositions.

### Usage Instructions

The present invention is also directed to a method of using any personal care composition comprising a water-disintegratable substrate and a lathering surfactant, including but not limited to the personal care compositions as described herein, wherein the method comprises the steps of (A) obtaining a personal cleansing composition comprising a water-disintegratable substrate and a lathering surfactant; and (B) providing instructions with the personal cleansing composition to perform the following usage steps: (i) lather the personal cleansing composition, (ii) wash the skin with the lathered composition, and then (iii) rinse the skin or hair. The method may further comprise instructions including a usage step of wetting or moistening the personal cleansing composition prior to the lathering instruction (i). The method may also further comprise instructions including a final usage step of disposing of any water-disintegratable substrate that remains after rinsing, preferably in a trash receptacle or down a drain.

The above-described usage instructions are preferably communicated by a picture or image sequence in which each of the usage instructions are visually displayed and thus effectively communicated to the user. Each picture sequence is preferably associated with text that describes each of the pictures in the sequence. For example, the usage instructions are highly effective when communicated as a sequence of 3-5 pictures with corresponding text such as 1) Wet, 2) Lather, 3) Wash, 4) Rinse, 5) Dispose, or combinations thereof, including usage steps 2-4, usage steps 1-4, usage steps 2-5, and so forth.

The above-described usage instructions can be any form such as text, visual pictures, or combinations thereof. The text instructions are preferably one word instructions, but may further comprise any number of other words to effectively communicate the particular usage step at issue.

### Method of Manufacture

The personal care compositions described herein may be prepared by any known or otherwise effective technique, suitable for making and formulating the desired or similar other product forms. Specific non-limiting examples of such methods as they are applied to specific embodiments of the present invention are described in the following examples. Preferred manufacturing methods of making the personal care compositions herein involve introducing a gas into a mixture of polymeric material and active ingredient comprising surfactant to make a foam, followed by drying of the resulting foamed material to a final moisture content. For example, such a process might include the following steps:

1. Heating and mixing a Cold Water Insoluble, Hot Water Soluble polymer and water until the polymer dissolves;

2. Blending a surfactant into the polymer solution,

3. Introducing gas into the resultant mixture, and adding energy, preferably mechanical energy by mixing, in order to make a wet foam mixture, and reduce the density to target wet mixture density value,

4. Adding optional ingredients to the mixture to increase the viscosity and/or render the polymer less water soluble and even completely insoluble,

5. Cooling the mixture, especially concurrent with the third and fourth steps, to increase the viscosity of the mixture in order to facilitate stabilization of the wet foam mixture against coalescence and drainage,

6. Forming the wet mixture into a desired shape, especially using a mold, and

7. Drying the wet mixture to a desired final moisture content, e.g., about 11% moisture, by addition of energy.

Optional ingredients can be added at any time during the above-described process. A preferred process sequence, however, involves adding a plasticizer to the polymer and water mixture during Step 1 to facilitate dissolution of the polymer. Optional ingredients are often added during the Step 4 process (e.g., addition of a cross-linking agent, physical or chemical, such as sodium bicarbonate or other carbonate when a polyvinyl alcohol or other similar polymer with a high degree of hydrolysis is used). Skin benefit agents, especially moisturizing agents, are preferably added during any of Steps 1-4, or can be coated, printed on, or laminated to the composition.

Step 1 may also be accomplished by subjecting a polymer mixture with the active ingredient to pressure and heat with a low level of water and/or cosolvents and/or plasticizer, such as in the barrel of an extruder, whereupon a significant amount of the water and cosolvent evaporate rapidly upon ejection from the extruder barrel, creating a stable foam that is dry or nearly dry. Such a mixture can be designated as a polymer melt, instead of a polymer solution. Air can also be injected into such a wet mixture, for example, in an extruder barrel.

An alternative manufacturing method for use herein involves spray-drying the wet polymer and active mixture in order to form aerated beads or particles which can be compressed such as in a mold with heat in order to form the solid polymeric foam component of the present invention.

In a preferred method of making the personal care compositions herein, the solid polymeric foam is prepared by a process comprising the steps of: (A) heating a liquid composition containing at least about 3% by weight of the polymeric structurant as solubilized or dispersed polymer in said liquid composition at a temperature of at least about 55° C.; (B) introducing air to the heated liquid composition; and (C) drying the air-containing composition to form the solid polymeric foam.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. All exemplified amounts are concentrations by weight of the total composition, i.e., wt/wt percentages, unless otherwise specified.

The personal care compositions described below all contain a high density, water-disintegratable, polymeric foam that provides topical delivery a surfactant or other active ingredient to the hair or skin, while only slowly disintegrating and rinsing away with water during prolonged single use or other similar application.

All of the exemplified compositions described hereinafter are applied topically to the hair and skin with water in accordance with the methods of the present invention described herein. In each instance, the exemplified composition is a substrate that is used in combination with warm or hot water (preferably about 104° F.) and rubbed over or otherwise moved across the applied surface repeatedly during a shower or bath lasting at least about 0.5 minutes, but not more than about 20 minutes, until the solid polymeric foam within each of substrates is partially or preferably completely disintegrated and rinsed away. The disintegrated substrates in each instance contains no individual foam pieces having a topographical area of more than about 10% of the original topographical area of the solid polymeric foam prior to application, and also no foam pieces having a topographical area of more than about 0.1 inch ², as determined in accordance with the Substrate Disintegration Test herein.

### Example 1

A surfactant paste premix is prepared. In a suitable vessel, the following ingredients are mixed at room temperature. Once the cationic guar polymer is dispersed, the mixture is heated to 65° C.

| Distilled water | | QS 100 |
|---|---|---|
| N Hance 3196 | cationic guar polymer | 0.450 |

While the mixture is being heated to 65° C. the following ingredients are added to the mixture.

| | | | |
|---|---|---|---|
| Hamposyl L30 | Sodium Lauroyl Sarcosinate | (30% active) | 25.500 |
| Empigen LCU | Lauramidopropyl Betaine | (35% active) | 22.000 |
| Aldrich | Citric acid, anhydrous | | 0.300 |
| Aldrich | Disodium EDTA | | 0.120 |
| Empicol ESB70 | Sodium Laureth2 Sulphate | (70% active) | 41.000 |
| Whittaker | Titanium Dioxide | | 0.350 |

Once the above ingredients are fully mixed, begin cooling the mixture to 45° C. In a separate mix vessel, add the following.

| | | |
|---|---|---|
| Distilled Water | | 3.000 |
| Kathon CG | | 0.030 |
| Aldrich | Sodium benzoate | 0.250 |

Once the preservatives are dissolved, add this mixture to the first mixing vessel and cool to room temperature. In a separate mix vessel, add the following and mix until homogeneous.

| | | |
|---|---|---|
| Cavamax W7 Pharma | Beta cyclodextrin | 2.500 |
| | Perfume | 0.500 |

Blend the mixture into the room temperature surfactant paste until homogeneous. (Cationic guar: Hercules Inc, Wilmington, Del.; Hamposyl L30: Hampshire Chemical, USA, 1-781-869-3433; Empigen LCU, Empicol ESB70: Huntsman Corp, Salt Lake City, Utah; Aldrich Chemical Co, Milwaukee, Wis.; Titanium dioxide: Whittaker, Clark and Daniels, Inc, Plainfield, Ill.; Kathon CG: Rohm & Haas, Spring House, Pa.; Cyclodextrin: Cerestar USA, Hammond, Ind.).

### Example 2

A surfactant paste premix is prepared. In a suitable vessel, the following ingredients are mixed at room temperature. Once the polyquaternium is dispersed, the mixture is heated to 65° C.

| Distilled water | QS 100 |
|---|---|
| Polyquaternium-20 | 1.00 |

While the mixture is being heated to 65° C. the following ingredients are added to the mixture.

| | |
|---|---|
| Ammonium Lauryl Sulfate | 5.60 |
| Ammonium Laureth Sulfate | 16.80 |
| Sodium Lauroamphoacetate | 9.60 |
| Disodium EDTA | 0.400 |
| Titanium Dioxide | 0.500 |

Once the above ingredients are fully mixed, begin cooling the mixture to 45° C. In a separate mix vessel, add the following.

| | |
|---|---|
| Distilled Water | 8.00 |
| Glydant Plus | 0.500 |
| Butylene Glycol | 8.00 |

Once the preservatives are dissolved, add this mixture to the first mixing vessel and cool to room temperature. Add perfume as necessary at room temperature and blend.

### Example 3

A surfactant paste premix is prepared which includes the following components.

| Component | Wt % |
|---|---|
| Decylpolyglucose | 12.0 |
| Cocamidopropyl betaine | 12.0 |
| Sodium lauroyl sarcosinate | 12.0 |
| Butylene glycol | 3.6 |
| PEG 14M | 1.8 |
| Polyquatemium-10 | 0.9 |
| Dex panthenol | 0.7 |
| Phenoxyethanol | 0.5 |
| Benzyl alcohol | 0.5 |
| Methylparaben | 0.45 |
| Propylparaben | 0.25 |
| Disodium EDTA | 0.2 |
| Water | 55.1 |

### Example 4

A tear-free liquid cleansing premix is prepared which includes the following components.

| Component | Wt % |
|---|---|
| Cocamido propyl betaine | 17.1 |
| Sodium trideceth sulfate | 8.3 |
| POE 100 sorbitan monooleate | 7.5 |
| Misc. (including perfume, preservative, dye) | 2.0 |
| Water | 65.1 |

Distinguishing characteristics of this composition are its non-irritating properties to skin and eyes.

### Example 5

A liquid cleansing premix is prepared which includes the following components.

| Component | | Wt % |
|---|---|---|
| Polyquaternium 10 | | 0.50 |
| Sodium Lauroamphoacetate | (27% active) | 20.0 |
| Sodium Laureth 3 Sulfate | (29% active) | 40.0 |
| Disodium EDTA | | 0.20 |
| Sodium citrate dihydrate | (88% active) | 0.60 |
| Citric acid, anhydrous | | 1.0 |
| PEG-6 caprylic/capric glycerides | | 2.0 |
| Cocamide MBA | (86% active) | 3.0 |
| Glycerin | | 3.5 |
| MgSO4-7H20 (Epsom salts) | | 1.5 |
| Maleated Soybean Oil | | 2.5 |
| Deodorized Soybean Oil | | 5.0 |
| Misc. (including perfume, caustic, colorant) | | 1.5 |
| Water | | 18.7 |

The mixture is mild for use on sensitive skin.

### Example 6

Soap shavings are prepared by shaving a bar soap which includes the following components:

| Component | Wt % |
|---|---|
| Sodium Cocyl Isethionate | 27.77 |
| Paraffin | 16.72 |
| Sodium Alkyl Glycerol Sulfonate (AGS) | 14.90 |
| Soaps | 11.41 |
| Glycerine | 8.57 |
| Water | 5.50 |
| Stearic Acid | 5.74 |
| Sodium Isethionate | 3.04 |
| NaCl | 1.41 |
| EDTA | 0.10 |
| Etidronic Acid | 0.10 |
| Polyox N-3000 PEG 14M | 0.03 |
| Perfume | 0.70 |
| Miscellaneous (including pigments) | 4.01 |
| Total | 100 |

### Example 7

A surfactant paste premix is prepared which includes the following components.

| Component | Wt % |
|---|---|
| EGDS | 3.1 |
| Cocamidopropyl betaine | 4.0 |
| TEA soap (Molecular Weight about 330) | 9.5 |
| Monoalkyl phosphate | 15.0 |
| Cocamine oxide | 7.5 |
| 1,2-propanediol | 1.0 |
| Ethanol | 3.0 |
| Miscellaneous (perfume, colorant, preservative) | 8.9 |
| Water | 48.0 |

Heat the mixture to 50 degrees Celsius, stirring continuously, until the mixture has lost 38% of its original weight, and it has a paste-like consistency.

### Example 8

Prepare a representative cleansing premix for the articles of the present invention in the following manner. Blend the cleansing component of Example 6 with 0.1% by weight of the bar soap flakes of a dermatologically acceptable enzyme which is preferably a keratinase, protease, amylase or subtilisin. Next, blend the resultant mixture with 2% by weight of the cleansing component of a dry hydrocolloid, sodium carboxymethylcellulose, and mill three times on a 3-roll mill. Store the enzyme cleansing premix in a suitable sealed container.

### Example 9

A liquid surfactant mixture is prepared. In a suitable vessel, the following ingredients are mixed. Heat the mixture to 65 degrees C.

| | Water | QS 100 |
|---|---|---|
| | Ammonium Lauryl Sulfate | 14.99 |
| Mirataine CBS | Cocamidopropyl hydroxysultaine | 7.50 |
| | Cyclohexane Dimethanol | 7.00 |
| | Disodium EDTA | 0.12 |
| | Sodium Hydroxide | 0.005 |

Once the above ingredients are fully mixed, begin cooling the mixture to 45° C. In a separate mix vessel, add the following.

| | | |
|---|---|---|
| | Distilled Water | 5.00 |
| Glydant | DMDM Hydantoin | 0.030 |
| | Sodium benzoate | 0.250 |

Once the preservatives are dissolved, add this mixture to the first mixing vessel and cool to room temperature. Add fragrance as needed at room temperature and mix until homogeneous.

(Mirataine CBS, Rhodia Inc, Cranbury, N.J.; Ammonium Lauryl Sulfate: Procter & Gamble Mexico, Mexico City; Cyclohexane Dimethanol: Eastman Kodak Co, Kingsport, Tenn.; Disodium EDTA, Sodium Hydroxide, Sodium Benzoate: Aldrich Chemical Co, Milwaukee, Wis.; Cyclohexane dimethanol: McIntyre Group Ltd, UK)

### Example 10

A lipid mixture is prepared. In a suitable vessel, the following ingredients are mixed. Heat the mixture to 75° C.

| | |
|---|---|
| Lanolin, anhydrous | 59.98 |
| Cetearyl methicone | 10.00 |
| C24-28 alkyl methicone | 5.00 |
| Petrolatum | 24.00 |
| CI 15850 Red 7 Calcium Lake in Castor Oil | 0.02 |

Once the above ingredients are fully mixed, begin cooling the mixture to 45° C. Add the following.

D,L-alpha tocopherol acetate 1.00

Once the mixture is homogeneous, cool to room temperature.

### Examples 11-13

In a stainless steel container, the following ingredients are mixed.

| | | Example | Example | Example |
|---|---|---|---|---|
| | | 11 | 12 | 13 |
| | Distilled water | QS 100 | QS 100 | QS 100 |
| Mowiol 2098 | Polyvinylalcohol | 17.44 | 18.40 | 18.67 |
| | Glycerin | 4.36 | 4.60 | 4.67 |

(Mowiol 2098: Clariant GMBH, Charlotte, NC; Glycerin, Aldrich Chemical Co.)

The mixture is heated using a heating mantle (GlasCol, Terre Haute, IN) to 85° C. and stirred rapidly but so as not to foam (e.g., with a Lightning Mixer manufactured by General Signal, Rochester, N.Y.) until all the polyvinyl alcohol is dissolved. The surfactant paste of Example 1 is added and mixed briefly until homogeneous.

Surfactant Paste 23.48 24.00 24.00

The mixture is transferred to a KitchenAid® (4½ Quart Standard Mixer bowl (KitchenAid, USA, 1-800-541-6390). The following ingredients are added.

| | | | |
|---|---|---|---|
| Sodium bicarbonate | 1.39 | 1.39 | 1.39 |
| Sodium carbonate | 1.39 | 1.39 | 1.39 |

(Sodium bicarbonate: Arm & Hammer brand; Sodium carbonate: Aldrich Chemical Co.)

The bicarbonate and carbonate are blended slowly for 10 seconds to disperse. The mixer is stopped and a spatula used to homogenize the mixture from top to bottom of the mixing bowl. The mixture is whipped on high speed until a wet mixture density of about 0.65 gm/cm³ (Example 11) is obtained. Example 12 is whipped until a wet mixture density of about 0.74 gm/cm³ is obtained, and Example 13 until a wet mixture density of about 0.81 gm/cm³ is obtained. Wet mixture density of the mixture is measured by weighing an aliquot in a beaker of known volume. The mixture is spread into a Teflon mold (“waffle mold”) comprising rectangular units, each unit comprising an array of 25 milled parallel rows by 15 milled parallel rows, each row 0.125 inches wide, with spacing of 0.125 inches between rows. Rows are milled to a depth of 0.0625 inches. The mixture is leveled in the mold using a straight edge and excess wet mixture removed. The filled mold is placed in an oven at 120° F. and dried until a moisture content of 11% is reached. The composition is removed from the mold and stored in a sealed polyethylene bag.

The composition of Example 11 has an Immersion Density of 0.53 gm/cm³, a Flash Lather Volume of 710 ml, a Total Lather Volume of 3,700 ml, a Dissolution Rate of 51%, a Basis Weight of 627 gsm, a Foam Thickness of 1.73 mm, and a Dry Drape of 49.6%. The composition of Example 12 has an Immersion Density of 0.57 gm/cm³, a Flash Lather Volume of 645 ml, a Total Lather Volume of 3,510 ml, a Dissolution Rate of 39%, a Basis Weight of 567 gsm, a Foam Thickness of 1.64 mm, and a Dry Drape of 18.7%. The composition of Example 13 has an Immersion Density of 0.67 gm/cm³, a Flash Lather Volume of 590 ml, a Total Lather Volume of 3,330 ml, a Dissolution Rate of 30%, a Basis Weight of 980 gsm, a Foam Thickness of 2.00 mm, and a Dry Drape of 2%.

The compositions are used to lather and cleanse the body in a shower. They lather for the duration of body cleansing, have a cloth-like feel and appearance, are mild to the skin, and leave the skin feeling very clean. Example 11 dissolves completely in only one shower. Example 12 is used to cleanse several children in one bath before it dissolves. Example 13 lathers for 2 consecutive showers before dissolving and/or dispersing in the rinse water.

### Example 14

In a stainless steel container, the following ingredients are mixed.

| | Distilled water | QS 100 |
|---|---|---|
| Mowiol 2098 | Polyvinylalcohol | 17.44 |
| | Glycerin | 2.86 |
| | Propylene Glycol | 1.50 |
| | Sodium chloride | 3.50 |

The mixture is heated using a heating mantle to 85° C. and stirred until all the polyvinyl alcohol is dissolved. The surfactant paste of Example 2 is added and mixed briefly until homogeneous.

Surfactant Paste 23.25

The mixture is transferred to a KitchenAid® 4½ Quart Standard Mixer. The following ingredients are added.

| | |
|---|---|
| Sodium bicarbonate | 0.60 |
| Sodium carbonate | 0.60 |

The bicarbonate and carbonate are blended slowly for 10 seconds to disperse. The mixer is stopped and a spatula used to homogenize the mixture from top to bottom of the mixing bowl. The mixture is whipped on high speed until a wet mixture density of about 0.65 gm/cm³ is obtained.

The mixture is spread into a Teflon mold comprising a 6 inch wide by 36 inch long groove milled to a depth of 0.030 inches. The mold has a ½ inch wide berm around each edge. The mixture is leveled in the molds using a straight edge and excess wet mixture removed. The filled mold is placed in an oven at 120° F. until partially dry, then placed under a 1500 Watt ceramic infrared heater (Salamander SWB/5, manufactured by Infared Internationale of North America, Ltd, Comstock Park, Mich.) until a moisture content of 11.0% is reached. The composition is removed from the molds and cut into 5.5 inch by 3 inch segments. Pairs of segments are laminated by lightly brushing one side with water using a paper towel, and using firm pressure for several minutes until sealed. The edges are finish cut. Approximately 100 holes, evenly spaced, are punched around the outer ⅝ inch of the segments. The holes encourage dissolution from the outside inward, upon exposure to water.

The composition has an Immersion Density of 0.52 gm/cm³, a Flash Lather Volume of 580 ml, a Total Lather Volume of 3,200 ml, a Dissolution Rate of 28%, a Basis Weight of 441 gsm, a Foam Thickness of 1.51 mm, and a Dry Drape of 40%.

### Examples 15-17

In the first step, in a stainless steel container, the following ingredients are mixed.

| | | Example | Example | Example |
|---|---|---|---|---|
| | | 15 | 16 | 17 |
| | Distilled water | QS 100 | QS 100 | QS 100 |
| Mowiol 2899 | Polyvinylalcohol | 15.70 | 18.40 | — |
| Mowiol 5698 | Polyvinylalcohol | — | — | 7.85 |
| Mowiol 383 | Polyvinylalcohol | — | — | 7.85 |
| | Glycerin | 1.76 | 4.60 | 1.76 |
| | 1,3-Butylene glycol | 0.87 | — | — |
| | Propylene glycol | 0.87 | — | — |
| | Cyclohexanedi- | — | — | 1.74 |
| | methanol | | | |

(Mowiol: Clariant GMBH, Charlotte, NC or Sigma Chemical Co.; Plasticizers, Sigma. or McIntyre Group Ltd, U.K.)

The mixture is heated using a heating mantle (GlasCol, Terre Haute, IN) to 95° C. and stirred vigorously but so as not to foam, by hand and with a mixer (e.g., with a Lightning Mixer manufactured by General Signal, Rochester, N.Y.) until all the polyvinyl alcohol is dissolved. Excess polyvinyl alcohol solution is prepared in the first step so as to transfer the correct amount of solution to the KitchenAid® mixer to maintain concentrations.

The mixture is transferred to a KitchenAid® 4½ Quart Standard Mixer bowl (KitchenAid, USA, 1-800-541-6390). The surfactant paste of Example 1 is added with the sodium bicarbonate and carbonate.

| | | | |
|---|---|---|---|
| Surfactant Paste | 19.87 | 24.00 | 19.87 |
| Sodium bicarbonate | 1.17 | 1.39 | 1.17 |
| Sodium carbonate | 1.17 | 1.39 | 1.17 |

(Sodium bicarbonate: Arm & Hammer brand; Sodium carbonate: Aldrich Chemical Co.)

The bicarbonate and carbonate are blended slowly for 10 seconds to disperse. The mixer is stopped and a spatula used to homogenize the mixture from top to bottom of the mixing bowl. The mixture is whipped on high speed until a wet mixture density of about 0.50 gm/cm³ (Example 15) is obtained. Example 12 is whipped until a wet mixture density of about 0.gm/cm³ is obtained, and Example 13 until a wet mixture density of about 0. gm/cm³ is obtained. Wet mixture density of the mixture is measured by weighing an aliquot in a beaker of known volume. The mixture of Examples 15 and 17 is spread into a Teflon mold (“waffle mold”) comprising rectangular units, each unit comprising an array of 25 milled parallel rows by 15 milled parallel rows, each row 0.125 inches wide, with spacing of 0.125 inches between rows. Rows are milled to a depth of 0.0625 inches. The mixture is leveled in the mold using a straight edge and excess wet mixture removed. 209.3 grams (Example 15) of the wet mixture is filled into the mold, and 20 grams are filled into a 200 mm diameter petri dish (Examples 15, 17). The wet mixture of Example 16 is spread into a Teflon waffle mold milled to a depth of 0.021 inches. The filled molds and petri dishes are placed in an oven at 120° F. and dried until a moisture content of about 11% is reached. The composition of Example 16 dries in about one-third the time of Examples 15 and 17. After removing from the mold, the composition of Example 16 is doubled by wetting the back slightly with water from a damp paper towel, pressing two halves together at the moist contact point, and applying pressure for about 2 minutes. The compositions are stored in sealed polyethylene bags.

The composition of Example 15 has an Immersion Density of 0.51 gm/cm³, a Flash Lather Volume of 780 ml, a Total Lather Volume of 3,680 ml, a Dissolution Rate of 42%, a Basis Weight of 370 gsm, a Foam Thickness of 2.15 mm, a Dry Drape of 32.8% (waffle molded composition) and a Wet Drape of 82.2%. The composition of Example 16 has an Immersion Density of 0.45 gm/cm³, a Flash Lather Volume of 690 ml, a Total Lather Volume of 3,580 ml, a Dissolution Rate of 48%, a Basis Weight of 363 gsm, a Foam Thickness of 2.09 mm, a Dry Drape of 26.1% and a Wet Drape of 84%.

The compositions are used to lather and cleanse the body in a shower. They lather for the duration of body cleansing, have a cloth-like feel and appearance, are mild to the skin, and leave the skin feeling very clean. Example 11 dissolves completely in only one shower. Example 12 is used to cleanse several children in one bath before it dissolves. Example 13 lathers for 2 consecutive showers before dissolving and/or dispersing in the rinse water.

### Example 18

In a stainless steel container, the following ingredients are mixed.

| | Distilled water | QS |
|---|---|---|
| | | 100 |
| Mowiol 5698 | Polyvinylalcohol | 10.65 |
| | Glycerin | 3.90 |

The mixture is heated using a heating mantle to 95° C. and stirred until most of the polyvinyl alcohol is dissolved. A Commercial Body Wash which comprises about 10% surfactants and about 85% water and which contains the following ingredients is added: water, sodium laureth sulfate, cocamidopropyl betaine, sodium sulfate, fragrance, sodium lauroyl sarcosinate, lauryl alcohol, DMDM hydantoin, tetrasodium EDTA, citric acid, Polyquaternium-10, D&C Red No. 33, D&C Green No. 5, FD&C Green No. 3 available as Zest Wild Sensations Body Wash, manufactured by the Procter & Gamble Co., Cincinnati, Ohio, USA. The heat and mixing is maintained until all the polyvinylalcohol is dissolved.

Commercial Body Wash 13.20

The mixture is transferred to a KitchenAid® 4½ Quart Standard Mixer. The following ingredients are added.

| | |
|---|---|
| Sodium bicarbonate | 0.70 |
| Sodium carbonate | 0.70 |

The bicarbonate and carbonate are blended slowly for 10 seconds to disperse. The mixer is stopped and a spatula used to homogenize the mixture from top to bottom of the mixing bowl. The mixture is whipped on high speed until a wet mixture density of about 0.56 gm/cm³ is obtained. The mixture is spread into a Teflon mold (“waffle mold”) comprising rectangular units, each unit comprising an array of 25 milled parallel rows by 15 milled parallel rows, each row 0.125 inches wide, with spacing of 0.125 inches between rows. Rows are milled to a depth of 0.0625 inches. The mixture is leveled in the mold using a straight edge. The filled mold is placed in a constant temperature environment room at 100F for 3 hours to dry while minimizing foam collapse, then in an oven at 120° F. until a moisture content of about 6% is reached. The composition is removed from the molds and stored in sealed polyethylene bags.

The composition has an Immersion Density of 0.235 gm/cm³, a Flash Lather Volume of 400 ml, a Total Lather Volume of 2,250 ml, a Dissolution Rate of 25%, a Basis Weight of 247 gsm, a Foam Thickness of 1.80 mm, and a Dry Drape of 82%.

### Example 19

A cleansing composition is prepared. In a stainless steel container, the following ingredients are mixed.

| | Distilled water | QS |
|---|---|---|
| | | 100 |
| Mowiol 2098 | Polyvinylalcohol | 16.10 |
| | Glycerin | 8.05 |

(Mowiol 2098: Clariant GMBH, Charlotte, NC; Glycerin, Aldrich Chemical Co.)

The mixture is heated using a heating mantle to 95° C. and stirred until all the polyvinyl alcohol is dissolved. A dispersion of laponite clay is added to stabilize the wet foam mixture. Add the following ingredient and stir until homogeneously mixed:

Laponite B 2% dispersion 20.12

(Laponite B 2% nanoclay dispersion in water from Southern Clay Products, Gonzales, Tex., 78629, USA)

The mixture is transferred to a KitchenAid® 4½ Quart Standard Mixer bowl (KitchenAid, USA, 1-800-541-6390). The surfactant paste of Example 7 is added.

Surfactant Paste 22.13

The mixture is whipped on high speed for 60 seconds until a wet mixture density of 0.44 gm/cm³ is obtained. Wet mixture density of the mixture is measured by weighing an aliquot in a beaker of known volume. The mixture is filled into a 100 mm diameter petri dishes which are placed in an oven at 120° F. and dried until a moisture content of about 18% is reached. The composition is removed from the dishes and stored in sealed polyethylene bags.

The composition has a Flash Lather Volume of 540 ml, a Total Lather Volume of 2,880 ml and a Dissolution Rate of 62%.

### Example 20

A fast-drying composition is prepared. In a stainless steel container in a well-ventilated hood, the following ingredients are mixed.

| | | |
|---|---|---|
| | Distilled water | 41.59 |
| Mowiol 2899 | Polyvinylalcohol | 14.70 |
| | Glycerin | 1.76 |
| | 1,3-Butylene glycol | 0.87 |
| | Propylene glycol | 0.87 |
| | n-Butanol | 5.00 |
| | Ethanol | QS |
| | | 100 |

(Mowiol 2899: Clariant GMBH, Charlotte, NC or Sigma Chemical Co.; Plasticizers and solvents, Sigma Chemical Co.)

The mixture is heated using a heating mantle (GlasCol, Terre Haute, IN) to 85-90° C. and stirred vigorously but so as not to foam, by hand and with a mixer (e.g., with a Lightning Mixer manufactured by General Signal, Rochester, N.Y.) until all the polyvinyl alcohol is dissolved. Excess polyvinyl alcohol solution is prepared in the first step so as to transfer the correct amount of solution to the KitchenAid® mixer to maintain concentrations.

The mixture is transferred to a KitchenAid® 4½ Quart Standard Mixer bowl (KitchenAid, USA, 1-800-541-6390). The surfactant paste of Example 1 is added with the sodium bicarbonate and carbonate.

| | |
|---|---|
| Surfactant Paste | 19.87 |
| Sodium bicarbonate | 1.17 |
| Sodium carbonate | 1.17 |

(Sodium bicarbonate: Arm & Hammer brand; Sodium carbonate: Aldrich Chemical Co.)

The bicarbonate and carbonate are blended slowly for 10 seconds to disperse. The mixer is stopped and a spatula used to homogenize the mixture from top to bottom of the mixing bowl. The mixture is whipped on high speed until a wet mixture density of about 0.50 gm/cm³ is obtained. Wet mixture density of the mixture is measured by weighing an aliquot in a beaker of known volume. The wet mixture is spread into a Teflon mold (“waffle mold”) comprising rectangular units, each unit comprising an array of 25 milled parallel rows by 15 milled parallel rows, each row 0.125 inches wide, with spacing of 0.125 inches between rows. Rows are milled to a depth of 0.0625 inches. The mixture is leveled in the mold using a straight edge and excess wet mixture removed. The filled mold is placed in a controlled temperature room at 120° F. and dried until a solvent content of about 11% is reached. The composition is stored in sealed polyethylene bags.

### Examples 21-23

In a stainless steel container, the following ingredients are mixed.

| | | Example | Example | Example |
|---|---|---|---|---|
| | | 21 | 22 | 23 |
| | Distilled water | QS | QS | QS |
| | | 100 | 100 | 100 |
| Mowiol 2098 | Polyvinylalcohol | 16.00 | 22.50 | 18.00 |
| | Glycerin | 4.00 | 7.50 | 4.50 |

(Mowiol 2098: Clariant GMBH, Charlotte, NC; Glycerin, Aldrich Chemical Co.)

The mixture is heated using a heating mantle (GlasCol, Terre Haute, IN) to 85° C. and stirred rapidly but so as not to foam (e.g., with a Lightning Mixer manufactured by General Signal, Rochester, N.Y.) until all the polyvinyl alcohol is dissolved. The surfactant paste of Example 1 (for Example 21) or Example 3 (for Examples 22 and 23) is added and mixed briefly until homogeneous.

Surfactant Paste 21.00 26.00 17.30

The following ingredients are added while continuing to maintain heat, and mixed until the mixture forms an oil-in-water emulsion and the mixture is homogeneous, about 5-7 minutes. The lipid mixture of Example 10 is used.

| | | | |
|---|---|---|---|
| Lipid mixture | 6.00 | 10.00 | 4.00 |
| Tego Pearl S33 | — | — | 1.00 |

(Tego Pearl S33: Goldschmidt Chemical Corp., Hopewell, VA 23860, USA)

The mixture is transferred to a KitchenAid® 4½ Quart Standard Mixer bowl (KitchenAid, USA, 1-800-541-6390). The following ingredients are added.

| | |
|---|---|
| Sodium bicarbonate | 1.30 |
| Sodium carbonate | 1.30 |

(Sodium bicarbonate: Arm & Hammer brand; Sodium carbonate: Aldrich Chemical Co.)

The bicarbonate and carbonate are blended slowly for 10 seconds to disperse. The mixer is stopped and a spatula used to homogenize the mixture from top to bottom of the mixing bowl. The mixture is whipped on high speed until a wet mixture density of about 0.72 gm/cm³ (Example 21) is obtained. Wet mixture density is measured by weighing an aliquot in a beaker of known volume. Examples 22 and 23 are whipped for 60 seconds. The mixture is spread into a Teflon mold (“waffle mold”) comprising rectangular units, each unit comprising an array of 25 milled parallel rows by 15 milled parallel rows, each row 0.125 inches wide, with spacing of 0.125 inches between rows. Rows are milled to a depth of 0.0625 inches. The mixture is leveled in the mold using a straight edge and excess wet mixture removed. The filled mold is placed in an oven at 120° F. and dried until a moisture content of about 10% is reached. The composition is removed from the mold and stored in a sealed polyethylene bag.

The composition of Example 21 has an Immersion Density of 0.64 gm/cm³, a Flash Lather Volume of 260 ml, a Total Lather Volume of 1,780 ml, a Dissolution Rate of 48%, a Basis Weight of 671 gsm, a Foam Thickness of 2.02 mm, a Dry Drape of 1% and a Wet Drape of 57%. The composition of Example 22 has an Immersion Density of 0.95 gm/cm³, a Flash Lather Volume of 110 ml, a Total Lather Volume of 490 ml, a Dissolution Rate of 28%, a Basis Weight of 1380 gsm, a Foam Thickness of 2.79 mm, a Dry Drape of 31% and a Wet Drape of 37%. The composition of Example 23 has an Immersion Density of 0.43 gm/cm³, a Flash Lather Volume of 160 ml, a Total Lather Volume of 1,200 ml, a Dissolution Rate of 93%, a Basis Weight of 395 gsm, a Foam Thickness of 1.99 mm, a Dry Drape of 62% and a Wet Drape of 84%.

The compositions are used to cleanse and condition the body in a shower. The composition of Example 21 lathers for the duration of body cleansing. The compositions of Examples 22 and 23 deliver skin conditioning while lathering to a lesser degree. The compositions have a cloth-like feel and appearance, are mild to the skin, deposit skin emollients, and leave the skin feeling clean.

### Example 24

In a stainless steel container, the following ingredients are mixed.

| | Distilled water | QS |
|---|---|---|
| | | 100 |
| Mowiol 2098 | Polyvinylalcohol | 14.30 |
| | Glycerin | 8.20 |
| | Sodium chloride | 1.02 |

(Mowiol 2098: Clariant GMBH, Charlotte, NC; Glycerin, Aldrich Chemical Co.)

The mixture is heated using a heating mantle (GlasCol, Terre Haute, IN) to 85° C. and stirred rapidly but so as not to foam (e.g., with a Lightning Mixer manufactured by General Signal, Rochester, N.Y.) until all the polyvinyl alcohol is dissolved. The surfactant flakes of Example 6 which contain skin emollients and titanium dioxide are added and mixed until the dispersion has no lumps.

Surfactant flakes 15.81

The mixture is transferred to a KitchenAid® 4½ Quart Standard Mixer bowl (KitchenAid, USA, 1-800-541-6390) and whipped on high speed for 80 seconds, until a wet mixture density of 0.46 gm/cm³ is obtained. The mixture is spread into a Teflon mold (“waffle mold”) comprising rectangular units, each unit comprising an array of 25 milled parallel rows by 15 milled parallel rows, each row 0.125 inches wide, with spacing of 0.125 inches between rows. Rows are milled to a depth of 0.0625 inches. The mixture is leveled in the mold using a straight edge and excess wet mixture removed. The filled mold is placed in an oven at 120° F. and dried until a moisture content of about 12% is reached. The composition is removed from the mold and stored in a sealed polyethylene bag.

The composition has an Immersion Density of 0.29 gm/cm³, a Calculated Density of 0.20 gm/cm³, a Flash Lather Volume of 230 ml, a Total Lather Volume of 1790 ml, a Dissolution Rate of 53%, a Basis Weight of 500 gsm, a Foam Thickness of 2.47 mm, and a Dry Drape of 9%.

### Example 25

In a stainless steel container, the following ingredients are mixed. The Moisturizing Body Wash of Example 5 is used.

| | Distilled water | QS |
|---|---|---|
| | | 100 |
| Mowiol 2098 | Polyvinylalcohol | 14.75 |
| | Glycerin | 4.61 |
| | Moisturizing Body Wash | 57.60 |

(Mowiol 2098: Clariant GMBH, Charlotte, NC; Glycerin, Aldrich Chemical Co.)

The mixture is heated using a heating mantle (GlasCol, Terre Haute, IN) to 90° C. and stirred rapidly but so as not to foam (e.g., with a Lightning Mixer manufactured by General Signal, Rochester, N.Y.) until all the polyvinyl alcohol is dissolved. The mixture is transferred to a KitchenAid® 4½ Quart Standard Mixer bowl (KitchenAid, USA, 1-800-541-6390) and whipped on high speed for 70 seconds, until a wet mixture density of 0.60 gm/cm³ is obtained. The mixture is spread into a Teflon mold (“waffle mold”) comprising rectangular units, each unit comprising an array of 25 milled parallel rows by 15 milled parallel rows, each row 0.125 inches wide, with spacing of 0.125 inches between rows. Rows are milled to a depth of 0.0625 inches. The mixture is leveled in the mold using a straight edge and excess wet mixture removed. The filled mold is placed in an oven at 120° F. and dried until a moisture content of about 2% is reached. The composition is removed from the mold and stored in a sealed polyethylene bag.

The composition has an Immersion Density of 0.50 gm/cm³, a Calculated Density of 0.30 gm/cm³, a Flash Lather Volume of 190 ml, a Total Lather Volume of 1200 ml, a Dissolution Rate of 24%, a Basis Weight of 709 gsm, a Foam Thickness of 2.38 mm, and a Dry Drape of 18%.

### Example 26

A 2-in-1 shampoo and conditioner which is also a body washing composition is prepared. In a stainless steel container, the following ingredients are mixed.

| | Distilled water | QS |
|---|---|---|
| | | 100 |
| Mowiol 2098 | Polyvinylalcohol | 18.00 |
| | Glycerin | 4.50 |
| | 2-in-1 shampoo & conditioner | 60.00 |
| | Magnesium chloride | 0.50 |

(Mowiol 2098: Clariant GMBH, Charlotte, NC; Glycerin, Magnesium chloride: Aldrich Chemical Co.)

The mixture is heated using a heating mantle (GlasCol, Terre Haute, IN) to 85° C. and stirred rapidly but so as not to foam (e.g., with a Lightning Mixer manufactured by General Signal, Rochester, N.Y.) until all the polyvinyl alcohol is dissolved. The 2-in-1 shampoo & conditioner is a commercial shampoo & conditioner sold under the name Suave For Kids 2 in 1 Shampoo and is tear free. The shampoo & conditioner contains: Water, Sodium Trideceth Sulfate, PEG-80 Sorbitan Laurate, Cocamidopropyl Hydroxysultaine, Disodium Lauroamphodiacetate, PEG-150 Distearate, Sodium Laureth-13 Carboxylate, Polysorbate 20, Glycerin, Polyquaternium-10, Citric Acid, Tetrasodium EDTA, DMDM Hydantoin, Fragrance, Methylchloroisothiazolinone, Methylisothiazolinone, FD&C Red No. 40, D&C Orange No. 4, and is manufactured by Helen Curtis, Chicago, Ill., 60610, USA.

The mixture is transferred to a KitchenAid® 4½ Quart Standard Mixer bowl (KitchenAid, USA, 1-800-541-6390) and whipped on high speed for 50 seconds, until a wet mixture density of 0.51 gm/cm³ is obtained. The mixture is spread into a Teflon mold (“waffle mold”) comprising rectangular units, each unit comprising an array of 25 milled parallel rows by 15 milled parallel rows, each row 0.125 inches wide, with spacing of 0.125 inches between rows. Rows are milled to a depth of 0.0625 inches. The mixture is leveled in the mold using a straight edge and excess wet mixture removed. The filled mold is placed in an oven at 120° F. and dried until a moisture content of about 10% is reached. The composition is removed from the mold and stored in a sealed polyethylene bag.

The composition has an Immersion Density of 0.35 gm/cm³, a Flash Lather Volume of 500 ml, a Total Lather Volume of 3,180 ml and a Dissolution Rate of 65.2%. The composition is used to lather and condition the hair in a shower, after which there is sufficient composition to lather and condition the body in the same shower.

### Example 27

The composition of Example 24 is prepared. The surfactant flakes of Example 8 are used in place of the Example 6 surfactant flakes.

### Example 28

An anti-acne and oil control composition is prepared. In a stainless steel container, the following ingredients are mixed. The Surfactant Paste of Example 1 is used.

| | Distilled water | QS |
|---|---|---|
| | | 100 |
| Mowiol 2098 | Polyvinylalcohol | 16.00 |
| | Glycerin | 4.00 |
| | Surfactant Paste | 14.40 |

(Mowiol 2098: Clariant GMBH, Charlotte, NC; Glycerin: Aldrich Chemical Co.)

The mixture is heated using a heating mantle (GlasCol, Terre Haute, IN) to 90° C. and stirred rapidly but so as not to foam (e.g., with a Lightning Mixer manufactured by General Signal, Rochester, N.Y.) until all the polyvinyl alcohol is dissolved. The mixture is transferred to a KitchenAid® 4½ Quart Standard Mixer bowl (KitchenAid, USA, 1-800-541-6390) and the following ingredients are added.

| | |
|---|---|
| 10% Benzoyl Peroxide lotion | 5.00 |
| Deep cleansing shower gel | 15.00 |

The 10% Benzoyl Peroxide lotion is Oxy Balance® Maximum Acne Treatment comprising 10% benzoyl peroxide with the following inactive ingredients: acrylates copolymer, carbomer 940, citric acid, diazolidinyl urea, dimethicones, dioctyl sodium sulfosuccinate, edetate disodium, glycerin, propylene glycol, silica, sodium citrate, sodium hydroxide, water, xanthan gum and is manufactured in Canada for GlaxoSmithKline Consumer Healthcare, L. P. in Pittsburgh, Pa., USA. The Deep Cleansing shower gel is Oxy Balance Deep Cleansing Shower Gel comprising 2% salicylic acid and contains the following inactive ingredients: cocamidopropyl betaine, decyl glucoside, disodium EDTA, FD&C Blue #1, fragrance, glycerin, hydroxycetyl hydroxyethyl dimonium chloride, lauryl polyglucose, PEG-40 hydrogenated castor oil, PEG-120 methyl glucose dioleate, phenoxyethanol, sodium hydroxide, trideceth 10, water.

The mixture is whipped on high speed until a wet mixture density of 0.53 gm/cm³ is obtained. The mixture is spread into a Teflon mold (“waffle mold”) comprising rectangular units, each unit comprising an array of 25 milled parallel rows by 15 milled parallel rows, each row 0.125 inches wide, with spacing of 0.125 inches between rows. Rows are milled to a depth of 0.0625 inches. The mixture is leveled in the mold using a straight edge and excess wet mixture removed. The filled mold is placed in an oven at 120° F. and dried until a moisture content of about 11% is reached. The composition is removed from the mold and stored in a sealed polyethylene bag.

The composition has an Immersion Density of 0.31 gm/cm³, a Flash Lather Volume of 460 ml, a Total Lather Volume of 2,460 ml and a Dissolution Rate of 75%.

### Example 29

A skin cleansing and conditioning composition is prepared. In a stainless steel container, the following ingredients are mixed.

| | Distilled water | QS |
|---|---|---|
| | | 100 |
| Mowiol 2098 | Polyvinylalcohol | 14.71 |
| | Glycerin | 3.68 |

(Mowiol 2098: Clariant GMBH, Charlotte, NC; Glycerin, Aldrich Chemical Co.)

The mixture is heated using a heating mantle (GlasCol, Terre Haute, IN) to 85° C. and stirred rapidly but so as not to foam (e.g., with a Lightning Mixer manufactured by General Signal, Rochester, N.Y.) until all the polyvinyl alcohol is dissolved. The mixture is transferred to a KitchenAid® 4½ Quart Standard Mixer bowl (KitchenAid, USA, 1-800-541-6390). The following ingredients are added. The Surfactant Paste of Example 1 is used.

| | |
|---|---|
| Surfactant paste | 17.23 |
| Sodium bicarbonate | 1.11 |
| Sodium carbonate | 1.11 |

(Sodium bicarbonate: Arm & Hammer brand; Sodium carbonate: Aldrich Chemical Co.)

The bicarbonate and carbonate are blended slowly for 10 seconds to disperse. The mixer is stopped and a spatula used to homogenize the mixture from top to bottom of the mixing bowl. The mixture is whipped on high speed for about 5 minutes, until a wet mixture density of about 0.42 gm/cm³ is obtained. The following liquid emollient is added:

SEFA cottonate 19.56

(SEFA cottonate: Sucrose Esters of Fatty Acids, fully esterified sucrose octaester with unhydrogenated cottonseed oil fatty acids, available from The Procter & Gamble Company, Ivorydale Manufacturing Plant, Cincinnati, Ohio, 45217, USA.)

The mixture is whipped on medium speed for about 4 minutes. The wet mixture density is 0.54 gm/cm³. The mixture is spread into a Teflon mold (“waffle mold”) comprising rectangular units, each unit comprising an array of 25 milled parallel rows by 15 milled parallel rows, each row 0.125 inches wide, with spacing of 0.125 inches between rows. Rows are milled to a depth of 0.0625 inches. The mixture is leveled in the mold using a straight edge and excess wet mixture removed. The filled mold is placed in an oven at 120° F. and dried until a moisture content of about 11% is reached. The composition is removed from the mold and stored in a sealed polyethylene bag.

The composition has an Immersion Density of 0.62 gm/cm³, a Flash Lather Volume of 610 ml, a Total Lather Volume of 3,370 ml and a Dissolution Rate of 75%.

### Example 30

A skin tanning and sun protection composition is prepared. In a stainless steel container, the following ingredients are mixed. The Surfactant Paste of Example 1 is used. The Sunscreen Lotion is a commercial sunscreen comprising octyl methoxycinnamate, octyl salicylate, oxybenzone, zinc oxide, aloe vera, cocoa butter and vitamin E and is manufactured by Solar Suncare, Miami Lakes, Fla. 33017, USA and sold under the name NO-AD (Not Advertised) SPF 45 Babies Maximum Sunblock.

| | Distilled water | QS |
|---|---|---|
| | | 100 |
| Mowiol 2098 | Polyvinylalcohol | 12.00 |
| Sigma 30-70,000 | Polyvinylalcohol | 5.00 |
| | Glycerin | 3.00 |
| | Surfactant Paste | 14.00 |
| | Sunscreen Lotion | 25.00 |

(Mowiol 2098: Clariant GMBH, Charlotte, NC; Glycerin, Aldrich Chemical Co.)

The mixture is heated using a heating mantle (GlasCol, Terre Haute, IN) to 85° C. and stirred rapidly but so as not to foam (e.g., with a Lightning Mixer manufactured by General Signal, Rochester, N.Y.) until all the polyvinyl alcohol is dissolved. The mixture is transferred to a KitchenAid® 4½ Quart Standard Mixer bowl (KitchenAid, USA, 1-800-541-6390). The following ingredients are added.

| | |
|---|---|
| Sunless Tanning Gel | 12.00 |
| Tocopherol nicotinate | 4.00 |

(Sunless Tanning Gel comprises water, glycerin, dihydroxyacetone, hydroxyethylcellulose, aloe vera gel, tocopheryl acetate, polysorbate 20, phenoxyethanol, methylparaben, ethylparaben, propylparaben, butylparaben, fragrance, phosphoric acid, sodium hydroxide and is manufactured by Solar Suncare, Miami Lakes, Fla. 33017, USA and sold under the name NO-AD (Not Advertised) Sunless Tanning Gel Medium-Dark Hypo-Allergenice Oil-Free; Tocopherol nicotinate: Sigma Chemical Co.)

The mixture is whipped on high speed until a wet mixture density of 0.87 gm/cm³ is obtained. The mixture is spread into a Teflon mold (“waffle mold”) comprising rectangular units, each unit comprising an array of 25 milled parallel rows by 15 milled parallel rows, each row 0.125 inches wide, with spacing of 0.125 inches between rows. Rows are milled to a depth of 0.0625 inches. The mixture is leveled in the mold using a straight edge and excess wet mixture removed. The filled mold is placed in an oven at 120° F. and dried until a moisture content of about 11% is reached. The composition is removed from the mold and stored in a sealed polyethylene bag.

The composition has an Immersion Density of 0.71 gm/cm³, a Flash Lather Volume of 110 ml, a Total Lather Volume of 440 ml and a Dissolution Rate of 58%. The composition is used in the shower after cleansing and rinsing the face and body have been completed by applying the composition to the skin, to condition and protect the face, hands and body against prospective sun damage and to artificially tan the skin.

### Example 31

A long lasting sweat and sebum absorbent composition is prepared. In a stainless steel container, the following ingredients are mixed. The Surfactant Paste of Example 1 is used.

| | Distilled water | QS |
|---|---|---|
| | | 100 |
| Mowiol 2098 | Polyvinylalcohol | 17.60 |
| | Glycerin | 4.40 |
| | Surfactant Paste | 26.00 |

(Mowiol 2098: Clariant GMBH, Charlotte, NC; Glycerin, Aldrich Chemical Co.)

The mixture is heated using a heating mantle (GlasCol, Terre Haute, IN) to 85° C. and stirred rapidly but so as not to foam (e.g., with a Lightning Mixer manufactured by General Signal, Rochester, N.Y.) until all the polyvinyl alcohol is dissolved. The following ingredients are separately premixed until the Bio-PSA is dissolved, then added to the mixture while continuing to maintain heat, and mixed until homogeneous, about 5 minutes.

| | |
|---|---|
| Bio-PSA Trimethylated silica | 5.82 |
| treated with dimethly siloxane | |
| Isopropyl palmitate | 5.82 |

(Bio-PSA is manufactured by Dow Corning Corp., Midland, MI, 48686, USA and sold under the name 7-4500 Silicone Adhesive Solids; Isopropyl palmitate: ISP Van Dyke, Belleville, NJ, 07109, USA.)

The following sweat and sebum absorbing particles are added, and the mixture is mixed until homogeneous.

Syloid 244 Hydrated silica 5.82

(Syloid 244: Davison Chemical division of W. R. Grace Co., New Castle, Del., 19720, USA).

The mixture is transferred to a KitchenAid® 4½ Quart Standard Mixer bowl (KitchenAid, USA, 1-800-541-6390) and whipped on high speed until a wet mixture density of 0.95 gm/cm³ is obtained. The mixture is spread into a Teflon mold (“waffle mold”) comprising rectangular units, each unit comprising an array of 25 milled parallel rows by 15 milled parallel rows, each row 0.125 inches wide, with spacing of 0.125 inches between rows. Rows are milled to a depth of 0.0625 inches. The mixture is leveled in the mold using a straight edge and excess wet mixture removed. The filled mold is placed in an oven at 120° F. and dried until a moisture content of about 10.6% is reached. The composition is removed from the mold and stored in a sealed polyethylene bag.

### Example 32

A reticulated foam is prepared, which is an antibacterial and antiviral composition. In a stainless steel container, the following ingredients are mixed with no heat at high agitation until the polymer is dispersed.

| | Distilled water | QS 100 |
|---|---|---|
| Polyox WSR-N3000 | PEG 14M | 0.030 |

(Polyox, Union Carbide)

The mixture is heated to 175° F. and then the following ingredients are added. Heating is continued to 195° F. and until all the polyvinylalcohol is dissolved.

| | | |
|---|---|---|
| Mowiol 2098 | Polyvinylalcohol | 11.94 |
| | Glycerin | 5.97 |

The mixture is transferred to a KitchenAid® 4½ Quart Standard Mixer. The following ingredients are mixed together, then added to the KitchenAid® mixer.

| | | |
|---|---|---|
| Aminoxid ® LO | 30% active Amine Oxide surfactant | 14.00 |
| Calfoam ES-703 | 70% active Sodium Laureth3 Sulfate | 3.01 |
| | Pyrrolidone Carboxylic Acid | 10.00 |

(Aminoxid ® LO: Goldschmidt Chemical Corp, Flopewell, VA 23860, USA; Sodium Laureth3 Sulfate: Pilot Chemical Co, Santa Fe Springs, CA 90670, USA; Pyrrolidone Carboxylic Acid: Aginimoto Chemicals, Japan)

Blend at slow speed until the surfactant mixture is incorporated into the polymer solution. Increase the mixing speed to maximum and mix about 5-10 minutes, until a chiffon-like texture results and wet mixture density of about 0.16 grams/cm³ is obtained. Add the following ingredients and blend slowly to incorporate without breaking the lipid spheres and capsules. If desired, conventional antibacterial ingredients such as triclosan can be added at this stage.

| | | |
|---|---|---|
| Lipospheres | 1500 micron petrolatum spheres, green | 5.0 |
| A2 Millicapsules | 3 mm petrolatum capsules, green | 5.0 |

Spread the mixture into a layer 2 cm thick on a cookie sheet and place the sheet in a controlled temperature room at 120° F. until a moisture content of about 6% is reached. The layer is cut into individual units about 4 inches long by 3 inches wide using curved cutting lines to fit the hand and improve ability to grip. The composition is used to lather and cleanse the body in a shower, especially to exfoliate, cleanse and moisturize the feet. It lathers for the duration of body cleansing, has a puff-like feel and appearance, is mild to the skin, exfoliates the skin, and leaves the skin feeling very clean.

The composition has a Foam Thickness of 5.71 mm, a Basis Weight of 1594 gsm, and a Calculated Density of 0.28 g/cm³.

### Example 33

A coacervate-forming composition which is a two-sided article is prepared. The first side is prepared as follows. In a stainless steel container, the following ingredients are mixed.

| | Distilled water | QS 100 |
|---|---|---|
| Monosodium lauroyl glutamate | | 20.0 |
| | Cocamidopropyl betaine | 2.0 |
| | Sodium chloride | 1.0 |
| | Glycerin | 2.5 |
| Mowiol 2098 | Polyvinylalcohol | 17.0 |

The mixture is heated using a heating mantle (GlasCol, Terre Haute, IN) to 85° C. and stirred rapidly but so as not to foam (e.g., with a Lightning Mixer manufactured by General Signal, Rochester, N.Y.) until all the polyvinyl alcohol is dissolved. The mixture is transferred to a KitchenAid® 4½ Quart Standard Mixer bowl (KitchenAid, USA, 1-800-541-6390). The following ingredients are added.

| | |
|---|---|
| Sodium bicarbonate | 0.98 |
| Sodium carbonate | 0.98 |

(Sodium bicarbonate: Arm & Hammer brand; Sodium carbonate: Aldrich Chemical Co.)

The bicarbonate and carbonate are blended slowly for 10 seconds to disperse. The mixer is stopped and a spatula used to homogenize the mixture from top to bottom of the mixing bowl. The mixture is whipped on high speed for about 5 minutes, until a wet mixture density of about 0.42 gm/cm³ is obtained. The mixture is spread into a Teflon mold (“waffle mold”) comprising rectangular units, each unit comprising an array of 25 milled parallel rows by 15 milled parallel rows, each row 0.125 inches wide, with spacing of 0.125 inches between rows. Rows are milled to a depth of 0.0625 inches. The mixture is leveled in the mold using a straight edge and excess wet mixture removed. The filled mold is placed in an oven at 120° F. and dried until a moisture content of about 11% is reached.

A second side is prepared as follows. In a stainless steel container, the following ingredients are mixed and heated until the polyvinylalcohol is dissolved.

| | Distilled water | QS 100 |
|---|---|---|
| Mowiol 2098 | Polyvinylalcohol | 16.75 |
| | Glycerin | 4.61 |
| | Moisturizing Body Wash | 8.85 |

The following ingredients are first heated to melt the lipid mixture, then blended together by hand stirring, then poured into the polyvinyl alcohol containing mixture. The Skin Anti-Aging Lotion is a commercial lotion containing the following ingredients: water, glycerin, distarch phosphate, C12-15 alkyl benzoate, sorbitan stearate, cyclomethicone, SD Alcohol 40-B, Biosaccharide Gum-1, cetearyl alcohol, polyglyceryl-3 methylglucose distearate, tocopheryl acetate, sodium ascorbyl phospohate, glucosylrutin (alpha flavon), isoquercitrin, fragrance, phenoxyethanol, citric acid, carbomer, xanthan gum, EDTA, sodium hydroxide, preservatives and iodopropynyl butylcarbamate, and is sold as Nivea Visage alpha flavone perfect protection, Manufactured by Beiersdorf Inc, Wilton, Conn., USA.

| | | |
|---|---|---|
| | Skin anti-aging lotion | 6.00 |
| Epomin SP-018 | Polyethyleneimine | 6.00 |

(Epomin SP-018, molecular weight about 1800, from Nippon Shokubai Co., Japan)

The mixture is stirred briefly until homogeneous, then transferred to a KitchenAid® 4½ Quart Standard Mixer bowl (KitchenAid, USA, 1-800-541-6390). The following ingredients are added.

The mixture is whipped on high speed for about 5 minutes, until a wet mixture density of about 0.76 gm/cm³ is obtained. The mixture is spread into a Teflon mold (“waffle mold”) comprising rectangular units, each unit comprising an array of 25 milled parallel rows by 15 milled parallel rows, each row 0.125 inches wide, with spacing of 0.125 inches between rows. Rows are milled to a depth of 0.0625 inches. The mixture is leveled in the mold using a straight edge and excess wet mixture removed. The filled mold is placed in an oven at 120° F. and dried until a moisture content of about 11% is reached.

Individual units of the first side and the second side are laminated together to form a composition that lathers by rubbing the first side against the skin in the presence of water, and forms a coacervate in the presence of the lather, depositing skin moisturization components on the skin when the second side is rubbed against the skin in the presence of water.

### Example 34

An anti-dandruff shampoo composition is prepared. In a stainless steel container, the following ingredients are mixed.

| | Distilled water | QS 100 |
|---|---|---|
| Mowiol 888 | Polyvinylalcohol | 12.00 |
| Mowiol 383 | Polyvinylalcohol | 4.00 |
| | Glycerin | 7.00 |
| | Antidandruff shampoo | 60.00 |

(Mowiol 888, 383: Clariant GMBH, Charlotte, NC)

The mixture is heated using a heating mantle (GlasCol, Terre Haute, IN) to 85° C. and stirred rapidly but so as not to foam (e.g., with a Lightning Mixer manufactured by General Signal, Rochester, N.Y.) until all the polyvinyl alcohol is dissolved. The antidandruff is a commercial shampoo sold under the name Head & Shoulders Dandruff Shampoo for Dry Scalp Care. The shampoo contains: Water, Ammonium Laureth Sulfate, Ammonium Lauryl Sulfate, Dimethicone, Sodium Chloride, Glycol Distearate, Cetyl Alcohol, Cocamide MEA, Fragrance, Polyquaternium-10, Sodium Citrate, Hydrogenated Polydecene, Sodium Benzoate, Trimethylolpropane Tricaprylate/Tricaprate, Citric Acid, Ammonium Xylenesulfonate, Ext. D&C Violet No. 2, FD&C Blue No. 1, Benzyl Alcohol, Methylchloroisothiazolinone, Methylisothiazolinone, and is manufactured by The Procter & Gamble Co., Cincinnati, Ohio, 45202, USA.

The mixture is transferred to a KitchenAid® 4½ Quart Standard Mixer bowl (KitchenAid, USA, 1-800-541-6390) and whipped on high speed for 50 seconds, until a wet mixture density of 0.50 gm/cm³ is obtained. The mixture is spread into a Teflon mold (“waffle mold”) comprising rectangular units, each unit comprising an array of 25 milled parallel rows by 15 milled parallel rows, each row 0.125 inches wide, with spacing of 0.125 inches between rows. Rows are milled to a depth of 0.0625 inches. The mixture is leveled in the mold using a straight edge and excess wet mixture removed. The filled mold is placed in an oven at 120° F. and dried until a moisture content of about 10% is reached. The composition is removed from the mold and stored in a sealed polyethylene bag. The individual units are cut in half prior to use as a shampoo product. The units dissolve in the hair very rapidly, lather effectively, and are used to treat or prevent dandruff. Shampoos containing other hair and scalp treating ingredients can also be used, for example Rogaine (Minoxidil).

### Example 35

An anti-cellulite treatment composition is prepared. In a stainless steel container, the following ingredients are mixed. The Surfactant Paste of Example 1 is used. The Anti-Cellulite Treatment is sold as RoC® Retinol Actif Pur® and comprises Water, Butylene Glycol, Cyclomethicone, Isononyl Isononanoate, Alcohol (2.73%), Glycerin, Caffeine, Dimethicone, Dimethicone Copolyol, Phenoxyethanol, Acrylates/C10-30 Alkyl Acrylate Crosspolymer, Shea Butter (Butyrospermum Parkii), Tromethamine, Glyceryl Stearate, PEG-100 Stearate, Dimethiconol, Methylparaben, BHT, Menthoxypropanediol, Disodium EDTA, Propylparaben, Ginkgo Biloba Extract, Butcherbroom (Ruscus Aculeatus) Extract, Polysorbate 20, Retinol, FD&C Yellow No. 6 and is manufactured by Johnson & Johnson

| | Distilled water | QS 100 |
|---|---|---|
| Mowiol 2098 | Polyvinylalcohol | 12.00 |
| Sigma 30-70,000 | Polyvinylalcohol | 5.00 |
| | Glycerin | 3.00 |
| | Surfactant Paste | 14.00 |
| | Anti-cellulite Treatment Lotion | 25.00 |

(Mowiol 2098: Clariant GMBH, Charlotte, NC; Sigma 30-70,000 Molecular Wt polymer: Sigma Chem. Co.; Glycerin, Aldrich Chemical Co.)

The mixture is heated using a heating mantle (GlasCol, Terre Haute, IN) to 85° C. and stirred rapidly but so as not to foam (e.g., with a Lightning Mixer manufactured by General Signal, Rochester, N.Y.) until all the polyvinyl alcohol is dissolved. The mixture is transferred to a KitchenAid® 4 1/2 Quart Standard Mixer bowl (KitchenAid, USA, 1-800-541-6390). The mixture is whipped on high speed until a wet mixture density of 0.68 gm/cm³ is obtained. The mixture is spread into a Teflon mold (“waffle mold”) comprising rectangular units, each unit comprising an array of 25 milled parallel rows by 15 milled parallel rows, each row 0.125 inches wide, with spacing of 0.125 inches between rows. Rows are milled to a depth of 0.0625 inches. The mixture is leveled in the mold using a straight edge and excess wet mixture removed. The filled mold is placed in an oven at 120° F. and dried until a moisture content of about 11% is reached. The composition is removed from the mold and stored in a sealed polyethylene bag.

The composition is used in the shower after cleansing and rinsing the body has been completed by applying the composition to the skin in the presence of water, to reduce the appearance of cellulite on the body.

### Example 36

The composition of Example 35 is prepared, substituting a commercial Lotion Hair Remover for the Anti-Cellulite Treatment. The commercial Lotion Hair Remover is Sally Hansen Lotion Hair Remover for difficult to remove hair, and comprises water, calcium thioglycolate, calcium hydroxide, urea, sodium silicate, cetearyl alcohol, potassium hydroxide, mineral oil, ceteareth-20, stearyl alcohol, tocopheryl acetate (vitamin E), anthemis nobilis flower oil, serenoa serrulata fruit extract, epilobium roseum oil, theobroma cacao (cocoa) seed butter, cocos nucifera (coconut) oil, aloe barbadensis leaf juice, bisabolol, dipotassium glycyrrhizate, fragrance, alcohol. The composition is used in the shower by applying to the legs, allowing the composition to stand on the legs, and rubbing during rinsing to remove hair. Shaving can also be accomplished at the same time.

### Example 37

A hair styling composition is prepared. In a stainless steel container, the following ingredients are mixed. The Hair Styling Gel is a commercial product which contains the following ingredients: Water, PVP, Glycerin, Polyacrylate-3, PEG-40 Hydrogenated Castor Oil, Fragrance, DMDM Hydantoin, Aminomethyl Propanol, Hydroxypropyltrimonium Hydrolyzed Wheat Protein, Iodopropynyl Butylcarbamate 03ACQD and is manufactured by L'Oreal USA, Inc., Dist. New York, N.Y., 10017 USA and sold as L'Oreal® Paris Curl Vive Curl-Shaping Spray Gel. The Surfactant Paste of Example 1 is used.

| | Distilled water | QS 100 |
|---|---|---|
| Mowiol 888 | Polyvinylalcohol | 12.00 |
| Mowiol 383 | Polyvinylalcohol | 4.00 |
| | Glycerin | 7.00 |
| | Surfactant Paste | 5.50 |
| | Hair Styling Gel | 55.00 |

(Mowiol 888, 383: Clariant GMBH, Charlotte, NC; Glycerin)

The mixture is heated using a heating mantle (GlasCol, Terre Haute, IN) to 85° C. and stirred rapidly but so as not to foam (e.g., with a Lightning Mixer manufactured by General Signal, Rochester, N.Y.) until all the polyvinyl alcohol is dissolved.

The mixture is transferred to a KitchenAid® 4½ Quart Standard Mixer bowl (KitchenAid, USA, 1-800-541-6390) and whipped on high speed for 50 seconds, until a wet mixture density of 0.46 gm/cm³ is obtained. The mixture is spread into a Teflon mold (“waffle mold”) comprising rectangular units, each unit comprising an array of 25 milled parallel rows by 15 milled parallel rows, each row 0.125 inches wide, with spacing of 0.125 inches between rows. Rows are milled to a depth of 0.0625 inches. The mixture is leveled in the mold using a straight edge and excess wet mixture removed. The filled mold is placed in an oven at 120° F. and dried until a moisture content of about 10% is reached. The composition is removed from the mold and stored in a sealed polyethylene bag. The individual units are cut in half prior to use as a shampoo product. The composition dissolves in the hair very rapidly in the presence of water, and is used to deposit hair styling polymer.

### Example 38

The cleansing composition of Example 12 is prepared. The individual units of the composition, which measure about 3.6 inches by 6.1 inches, are mated and sealed to a similar size piece of a batting composition. The batting composition has a basis weight of 4 oz/yd 2 and is comprised of polyester fibers of about 30 microns average diameter and is adhesive bonded, available for example as Mountain Mist Extra Heavy Batting #205 from Stearns Textiles, Cincinnati, Ohio. The seal is effected by moistening one side of the composition of Example 12 with an adhesive solution of 12% Mowiol 2098 polyvinylalcohol and 4% glycerin in 70/30 water/ethanol, compressing the batting against the moist composition with a pressure of about 3 psi for one-half hour, removing the compressive force, and placing the sealed composition at 120° F. for about one-half hour to return it to its original moisture content.

### Example 39

A two-sided, lathering and conditioning composition is prepared. The lathering and cleansing composition of Example 11 is prepared, and the conditioning composition of Example 22 is prepared. Individual units of each composition, which measure about 3.6 inches by 6.1 inches, are bonded to each other using the bonding method described in Example 38 to create an article that lathers preferably when one side is applied to the skin in the presence of water, and conditions preferably when the second side is applied to the skin in the presence of water. Instructions are provided to a user to rub first the lathering side against the skin, followed by the conditioning side. As the lathering side is depleted during use, due to its faster dissolution rate profile and use of the lathering side first against the skin, the cloth transitions to a conditioning article, thus achieving almost a two-step profile in a single composition.

### Example 40

The two-sided article of Example 39 is prepared except that bonding is effected using an ultrasonic sealer which seals a dot pattern comprising a grid of 4 mm diameter sealing points spaced evenly across the composition at 3 cm intervals.

### Example 41

A multi-layered article is prepared. The lathering and cleansing composition of Example 11 is prepared and is bonded to a conventional nonwoven substrate, which is Chicopee 9931, a 50/50 rayon/polyester carded, hydroentangled, apertured nonwoven with a basis weight of 62 gsm manufactured by Chicopee, Inc., New Brunswick, N.J., USA. The conditioning composition of Example 22 is prepared, and is cut into one-inch wide strips. A one-inch wide strip of the conditioning composition is bonded to the conventional nonwoven substrate on the side opposite to the lathering and cleansing composition. The article is used by lathering with one side against the skin or hair followed by conditioning with the other side against the skin or hair.

### Example 42

A cleansing and conditioning article is prepared. The cleansing composition of Example 12 is prepared. The lipid mixture of Example 10 is prepared and is heated to 70° C. in a hot melt coating reservoir tank. The lipid is slot coated in a 1 inch wide strip across the length of the composition of Example 12 at a coating weight of about 380 gsm which is a Foam Thickness of about 0.4 mm. The article is used by cleansing the skin with one side and conditioning the skin using the side with the lipid stripe.

### Example 43

The reticulated foam composition of Example 32 is prepared. Instead of petri dish molds, the wet mixture is coated onto a conventional nonwoven substrate, which is a cellulose paper towel having sufficient wet strength to last through a single use in the shower. The paper towel is an adhesive bonded cellulose paper towel with good loft and a basis weight of about 53 gsm. A useful towel is available from The Procter & Gamble Company and marketed as Bounty Rinse & Reuse®, which retains its Z-direction height when wet, and which has a Foam Thickness of about 0.047 inches at 5 gsi. A segment of substrate is stretched across a cookie sheet and taped on all sides. The 1.25 cm shims of Example 50 are placed 8 inches apart, parallel, on top of the taped substrate. The wet mixture is coated onto the taped substrate surface, and drawn with a straight edge blade to create a level, flat top surface of the wet mixture. The cookie sheet is placed at a constant temperature of 120° F. and dried to a moisture content of about 11%. The composition is cut into 6 inch by 4 inch articles and stored in a polyethylene bag. The composition is entirely biodegradable and can even be flushed into most septic systems due to its size and biodegradability.

### Example 44

A reticulated foam composition is prepared. In a stainless steel container, the following ingredients are mixed with no heat at high agitation until the polymer is dispersed.

| | Distilled water | QS 100 |
|---|---|---|
| Polyox WSR-N3000 | PEG 14M | 0.030 |

(Polyox, Union Carbide)

The mixture is heated to 175° F. and then the following ingredients are added. Heating is continued to 195° F. and until all the polyvinylalcohol is dissolved.

| | | |
|---|---|---|
| Mowiol 2098 | Polyvinylalcohol | 11.94 |
| | Glycerin | 5.97 |

The mixture is transferred to a KitchenAid® 4½ Quart Standard Mixer. The liquid surfactant mixture of Example 9 is added.

Surfactant mixture 26.11

Blend at slow speed until the surfactant mixture is incorporated into the polymer solution. Increase the mixing speed to maximum and mix about 5-10 minutes, until a chiffon-like texture results and wet mixture density of about 0.1 grams/cm³ is obtained. Spread the mixture into 100 mm petri dishes, for example Falcon Optilux® 100×20 mm Style 351005 plastic petri dished manufactured by Becton Dickinson and Co., Franklin Lakes, N.J., USA. Place the filled dishes in a still air oven at 140° F. until a moisture content of about 5% is reached.

The composition has a Flash Lather Volume of 740 ml, a Total Lather Volume of 3,310 ml, a Dissolution Rate of 51%, a Basis Weight of 372 gsm, a Foam Thickness of 5.91 mm, and a Calculated Density of 0.063 grams/cm³.

The composition is laminated to a layer of the composition of Example 31, which has an Immersion Density of 0.95 gm/cm³, a Flash Lather Volume of 110 ml, a Total Lather Volume of 490 ml, a Dissolution Rate of 28%, a Basis Weight of 1380 gsm, a Foam Thickness of 2.79 mm, a Dry Drape of 31% and a Wet Drape of 37%.

The laminated composition is used to lather and cleanse the body in a shower using the reticulated foam side against the skin. It lathers for the duration of body cleansing, has a puff-like feel and appearance, is mild to the skin, exfoliates the skin, and leaves the skin feeling very clean. After cleansing, the laminated composition is turned over and the second side is used to apply skin moisturization.

### Example 45

Part A: In a stainless steel container, the following ingredients are mixed.

| | Distilled water | QS 100 |
|---|---|---|
| Hercules USP/200 | Pectin | 9.36 |
| | Glycerin | 4.90 |

(Hercules USP/200 Pectin: Hercules, Inc. Wilmington, DE 19894; Glycerin, Aldrich Chemical Co.)

The mixture is heated using a heating mantle (GlasCol, Terre Haute, IN) to 80° C. and stirred slowly so as not to foam (e.g., with a Lightning Mixer manufactured by General Signal, Rochester, N.Y.) until all the pectin is dissolved.

Part B: In a separate container, the following ingredients are mixed and heated on a hot plate to about 75° C.

| | | |
|---|---|---|
| | Distilled water | 20.00 |
| Hercules 0667-56-01 | Cationic guar, medium MWt. | 0.41 |

The mixture of Part A is transferred to a KitchenAid® 4½ Quart Standard Mixer bowl (KitchenAid, USA, 1-800-541-6390). The following ingredient is added. The surfactant paste of Example 1 is used.

Surfactant Paste 12.27

The mixture is whipped on high speed using the KitchenAid® whisk attachment about 30 seconds, until a foam develops. The mixture of Part B is then added to the foam while the whipping is taking place, and whipping continues until a wet mixture density of about 0.50 gm/cm³ is obtained. Wet mixture density of the mixture is measured by weighing an aliquot in a beaker of known volume. The mixture is spread into a Teflon mold (“waffle mold”) comprising rectangular units, each unit comprising an array of 25 milled parallel rows by 15 milled parallel rows, each row 0.125 inches wide, with spacing of 0.125 inches between rows. Rows are milled to a depth of 0.0625 inches. The mixture is leveled in the mold using a straight edge and excess wet mixture removed. The filled mold is placed in an oven at 120° F. and dried until a moisture content of 14% is reached. The composition is removed from the mold and stored in a sealed polyethylene bag.

The composition has a Flash Lather Volume of 470 ml, a Total Lather Volume of 2,530 ml and a Dissolution Rate of 100%.

### Example 46

In a stainless steel container, the following ingredients are mixed.

| | Distilled water | QS 100 |
|---|---|---|
| Mowiol 2098 | Polyvinylalcohol | 14.40 |
| | Glycerin | 4.50 |
| Argo | Cornstarch | 3.60 |

(Mowiol 2098: Clariant GMBH, Charlotte, NC; Glycerin, Aldrich Chemical Co.; Argo Cornstarch: commercial cornstarch)

The mixture is heated using a heating mantle (GlasCol, Terre Haute, IN) to 90-95° C. and stirred slowly so as not to foam (e.g., with a Lightning Mixer manufactured by General Signal, Rochester, N.Y.) until all the polyvinyl alcohol is dissolved and the cornstarch gelatinizes. The mixture is transferred to a KitchenAid® 4½ Quart Standard Mixer bowl (KitchenAid, USA, 1-800-541-6390). The following ingredients are added. The surfactant paste of Example 1 is used.

| | |
|---|---|
| Surfactant Paste | 19.50 |
| Sodium carbonate | 0.50 |

(Sodium carbonate: Sigma Chemical Co.)

The mixture is whipped on high speed until a wet mixture density of about 0.52 gm/cm³ is obtained. Wet mixture density of the mixture is measured by weighing an aliquot in a beaker of known volume. The mixture is spread into a Teflon mold (“waffle mold”) comprising rectangular units, each unit comprising an array of 25 milled parallel rows by 15 milled parallel rows, each row 0.125 inches wide, with spacing of 0.125 inches between rows. Rows are milled to a depth of 0.0625 inches. The mixture is leveled in the mold using a straight edge and excess wet mixture removed. The filled mold is placed in an oven at 120° F. and dried until a moisture content of 9% is reached. The composition is removed from the mold and stored in a sealed polyethylene bag.

The composition has an Immersion Density of 0.29 gm/cm³, a Calculated Density of 0.11 gm/cm³, a Flash Lather Volume of 570 ml, a Total Lather Volume of 3,170 ml, a Dissolution Rate of 98%, a Basis Weight of 255 gsm, a Foam Thickness of 2.28 mm and a Dry Drape of 57%.

### Example 47

In a stainless steel container, the following ingredients are mixed. The surfactant paste of Example 1 is used.

| | Distilled water | QS 100 |
|---|---|---|
| Mowiol 2098 | Polyvinylalcohol | 7.22 |
| | Glycerin | 1.88 |
| | Surfactant Paste | 8.92 |

(Mowiol 2098: Clariant GMBH, Charlotte, NC; Glycerin, Aldrich Chemical Co.)

The mixture is heated using a heating mantle (GlasCol, Terre Haute, IN) to 80° C. and stirred slowly so as not to foam (e.g., with a Lightning Mixer manufactured by General Signal, Rochester, N.Y.) until all the polyvinyl alcohol is dissolved. After the polyvinyl alcohol is dissolved, the mixture is removed from the heating mantle while continuing to stir, and the latex emulsion polymer mixture is added by pouring into the vortex. The latex emulsion polymer is a commercial acrylic latex comprising water, acrylic resin emulsion, titanium dioxide, calcium carbonate and ethylene glycol and is manufactured by Masterchem® Industries, Inc., Barnhart, Mo. 63012, USA and is sold under the name Kilz® Total One® water-base Sealer-Primer-Stainblocker.

Latex emulsion polymer mixture 17.90

After mixing briefly until homogeneous, the mixture is transferred to a KitchenAid® 4½ Quart Standard Mixer bowl (KitchenAid, USA, 1-800-541-6390). The following ingredients are added. The surfactant paste of Example 1 is used.

Surfactant Paste 8.92

Sigma 30-70,000 CWS Polyvinylalcohol 8.22

(Sigma 30-70,000 Cold Water Soluble Polyvinylalcohol: Sigma Chemical Co.)

The mixture is blended for about 2 minutes on low speed, stirring occasionally by hand. The following ingredients are added while the KitchenAid mixer is whipping:

| | |
|---|---|
| Sodium bicarbonate | 1.50 |
| Sodium carbonate | 1.50 |

(Sodium bicarbonate: Arm & Hammer brand; Sodium carbonate: Aldrich Chemical Co.)

The mixture is whipped on high speed until a wet mixture density of about 0.52 gm/cm³ is obtained. Wet mixture density of the mixture is measured by weighing an aliquot in a beaker of known volume. The mixture is spread into a Teflon mold (“waffle mold”) comprising 122 mm by 152 mm rectangular units, each unit comprising an array of 20 by 25 evenly spaced rows of nubs, each nub comprising a ⅛ inch diameter vertical cylinder which is the unmilled portion of Teflon after the area between the nubs has been milled. The three outside rows are entirely populated with nubs; the next three interior rows are populated only with half the nubs (the other half milled to the same depth as the neighboring area); the remaining interior portion of the mold has only eighteen total nubs. The area between the nubs is milled to a depth of 1/16 inch at the outside edge, gradually increasing to a depth of ⅛ inch in the center. The mixture is leveled in the mold using a straight edge and excess product removed. The filled mold is placed in an oven at 120° F. and dried until a moisture content of 9% is reached. The product is removed from the mold and stored in a sealed polyethylene bag.

The composition has an Immersion Density of 0.43 gm/cm³, a Calculated Density of 0.28 gm/cm³, a Flash Lather Volume of 450 ml, a Total Lather Volume of 2,710 ml, a Dissolution Rate of 28%, a Basis Weight of 604 gsm, a Foam Thickness of 2.18 mm, a Dry Drape of 22% and a Wet Drape of 75%.

### Example 48

The composition of Example 11 is prepared. The wet mixture is spread into Teflon mold having the same dimensions of the rows, except that the mold used has continuous rows from side to side and end to end with no separation into discrete units. A continuous strip of composition about 36 inches long is thus prepared, and dried to a moisture content of about 14%. After drying, a serrated razor knife cutting blade cuts a serrated pattern at about 2.5 inch intervals along the length of the strip. The strip is rolled onto a 1 inch diameter plastic bar and set into a holder which allows the bar to rotate freely. A housing covers the composition and holder assembly, which has a slot through which the composition sheet is fed. The composition and housing are designed for mounting or hanging in the shower so that by tearing along the serration, units of composition are dispensed. Size is selected by choosing to tear off one or more than one unit.

### Example 49

In a stainless steel container, the following ingredients are mixed.

| | Distilled water | QS 100 |
|---|---|---|
| Mowiol 1098 | Polyvinyl alcohol | 7.54 |
| Mowiol 888 | Polyvinyl alcohol | 4.00 |
| Mowiol 383 | Polyvinyl alcohol | 3.00 |
| | Glycerin | 4.36 |

(Mowiol 1098, 888, 383: Clariant GMBH, Charlotte, NC; Glycerin, Aldrich Chemical Co.)

The mixture is heated using a heating mantle (GlasCol, Terre Haute, IN) to 85° C. and stirred rapidly but so as not to foam (e.g., with a Lightning Mixer manufactured by General Signal, Rochester, N.Y.) until all the polyvinyl alcohol is dissolved. The surfactant paste of Example 1 is added and mixed briefly until homogeneous.

Surfactant Paste 26.98

The mixture is transferred to a KitchenAid® 4½ Quart Standard Mixer bowl (KitchenAid, USA, 1-800-541-6390). The following ingredients are added.

| | |
|---|---|
| Sodium bicarbonate | 0.99 |
| Sodium carbonate | 0.29 |

(Sodium bicarbonate: Arm & Hammer brand; Sodium carbonate: Aldrich Chemical Co.)

The bicarbonate and carbonate are blended slowly for 10 seconds to disperse. The mixer is stopped and a spatula used to homogenize the mixture from top to bottom of the mixing bowl. The mixture is whipped on high speed until a wet mixture density of about 0.61 gm/cm³ is obtained. Wet mixture density of the mixture is measured by weighing an aliquot in a beaker of known volume. The mixture is spread into a Teflon mold (“waffle mold”) comprising rectangular units, each unit comprising an array of 25 milled parallel rows by 15 milled parallel rows, each row 0.125 inches wide, with spacing of 0.125 inches between rows. Rows are milled to a depth of 0.0625 inches. The mixture is leveled in the mold using a straight edge and excess wet mixture removed. The filled mold is placed in an oven at 120° F. and dried until a moisture content of 11% is reached. The composition is removed from the mold and stored in a sealed polyethylene bag, one individual unit per bag.

The composition is used to cleanse a dog. The composition lathers well and dissolves quickly even in cold water.

What is claimed is:

1. A method of topically applying active ingredients to the hair or skin, said method comprising the steps of:
(A) applying to the hair or skin a combination of water and a personal care composition, said composition comprising a high-density solid polymeric foam having Foam Density of from about 0.23 grams/cm³ to about 1.20 grams/cm³, said polymeric foam comprising:
(i) a solid polymer structurant; and
(ii) an active ingredient comprising a surfactant in a weight ratio of the surfactant to the solid polymer structurant of from about 1:8 to about 10:1; and
(B) rinsing the hair or skin with water to disintegrate and rinse-away the high-density polymeric foam such that the disintegrated polymeric foam contains no individual foam pieces having a topographical area of more than about 10% of the original topographical area of the solid polymeric foam prior to application in accordance with the Substrate Disintegration Test.

2. The method of claim 1, wherein the solid polymeric structurant is a synthetic polymer.

3. The method of claim 1, wherein the solid polymeric foam is prepared by a process comprising the steps of:
(A) heating a liquid composition containing at least about 3% by weight of the polymeric structurant as solubilized or dispersed polymer in said liquid composition at a temperature of at least about 55° C.;
(B) introducing air to the heated liquid composition; and
(C) drying the air-containing composition to form the solid polymeric foam.

4. The method of claim 1 wherein the Foam Density is an Immersion Density of from about 0.23 grams/cm³ to about 0.7 grams/cm³.

5. The method of claim 1 wherein the Foam Density is a Calculated Density of from about 0.25 grams/cm³ to about 0.7 grams/cm³.

6. The method of claim 1 wherein the disintegrated polymeric foam contains no individual foam pieces having a topographical area of more than 0.10 inch ² as determined by the Substrate Disintegration Test.

7. The method of claim 1 wherein the personal care composition is in the form of a thin sheet having a Foam Thickness of from about 0.2 mm to about 40 mm.

8. The method of claim 1 wherein the personal care composition is in the form of a thin sheet having a Foam Thickness of from about 1.5 mm to about 7 mm.

9. The method of claim 1 wherein the solid polymeric foam has a basis weight of from about 250 grams/² to about 1,000 grams/m².

10. The method of claim 1 wherein the solid polymeric foam has a basis weight of from about 50 grams/m² to about 3,000 grams/m².

11. The method of claim 1 wherein the solid polymeric foam has a Dry Drape Value of from zero to about 84.

12. The method of claim 1 wherein the solid polymeric foam has a Dry Drape Value of from about 30 to about 80.

13. The method of claim 1 wherein the solid polymeric foam has a Wet Drape Value of from about 5 to about 84.

14. The method of claim 1 wherein the solid polymeric foam has a Wet Drape Value of from about 40 to about 84.

15. The method of claim 1 wherein the solid polymeric foam has a Total Lather Volume of from about 1,000 ml to about 4,000 ml.

16. The method of claim 1 wherein the solid polymeric foam has a Total Lather Volume of from about 2,000 ml to about 4,000 ml.

17. The method of claim 1 wherein the solid polymeric foam has a Total Lather Volume of from about 400 ml to about 1,000 ml.

18. The method of claim 1 wherein the solid polymeric foam has a Total Lather Volume of from about 400 ml to about 700 ml.

19. The method of claim 1 wherein the solid polymeric foam has a Dissolution Rate of from about 10% to about 100%.

20. The method of claim 1 wherein the solid polymeric foam has a Dissolution Rate of from about 27% to about 80%.

21. The method of claim 1 wherein the solid polymer foam has a Moisture Retention of less than about 0.i grams.

22. The method of claim 1 wherein the solid polymer foam has a Moisture Retention of from about

23. The method of claim 1 wherein the surfactant represents from about 0.5% to about 75% by weight of the solid polymeric foam.

24. The method of claim 1 wherein the surfactant represents from about 1% to about 50% by weight of the solid polymeric foam.

25. The method of claim 1 wherein the surfactant comprises an anionic cleansing surfactant.

26. The method of claim 1 wherein the solid polymeric foam further comprises a plasticizing agent.

27. The method of claim 25 wherein the plasticizing agent is selected from the group consisting of glycerol, propylene glycol, butylenes glycol, cyclohexane dimethanol, alkyl phthallates, allyl phthallates, napthalates, ethyl acetate, butyl acetate, benzyl alcohol, amyl acetate, propyl acetate, acetone, heptane, iso-butyl acetate, iso-propyl acetate, toluene, methyl acetate, iso-butanol, n-amyl alcohol, n-butyl alcohol, hexane, methanol, ethanol, n-propanol, isopropanol, n-butanol, isobutanol, methylethylketone, acetone, and combinations thereof.

28. The method of claim 1 wherein the solid polymer structurant is hot water soluble and cold water insoluble as characterized by a viscosity of less than about 15 cP as determined by the Cold Water Insolubility Test and a viscosity of greater than about 10 cP as determined by the Hot Water Solubility Test.

29. The method of claim 1 wherein the solid polymer structurant is characterized by a viscosity of less than about 15 cP as determined by a Cold Water Insolubility Test and a viscosity of greater than about 15 cP as determined by a Second Cold Water Insolubility Test.

30. The method of claim 1 wherein the personal care composition is a shampoo composition intended for application to the hair.

31. The method of claim 1 wherein the personal care composition is a body cleansing composition intended for application to the skin.

32. The method of claim 1, wherein the solid polymer structurant is selected from the group consisting of polyvinyl alcohols, polyvinyl pyrrolidone, polyalkylene oxides, celluloses, cellulose derivatives, polysaccharides, polysaccharide derivatives, polycarboxylic acids, salts of polycarboxylic acids, polyamino acids, peptides, polyamides, polyacrylamides, polyesters, poly(vinyl methyl ether-co-maleic anhydride), alginates, alginate derivatives, pectins, polyethylene oxides, gelatins, carrageenans, chitosans, starches, starch derivatives, and combinations thereof.

33. The method of claim 1 wherein the solid polymer structurant comprises polyvinyl alcohol.

34. The method of claim 1 wherein the water has an average temperature of at least about 85° F.

35. The method of claim 1 wherein the water has an average temperature of from about 100° F. to about 140° F.

36. The method of claim 1 wherein the composition is applied during a bath or shower lasting for at least about 0.5 minutes.

37. The method of claim 1 wherein the composition is applied during a bath or showering lasting from about 2 minutes to about 20 minutes.

38. The method of claim 1 wherein the ratio of the surfactant to the solid polymer structurant is from about 1:1.6 to about 10:1.

39. The method of claim 1 wherein the composition is in the form of a textured substrate.

40. A method of using a personal cleaning composition on the hair or skin, said method comprising the steps of:
(A) obtaining a personal cleansing composition comprising a water-disintegratable substrate and a lathering surfactant; and
(B) providing instructions with the personal cleansing composition to perform the following usage steps:
(i) lather the personal cleansing composition,
(iii) wash the skin or hair with the lathered composition, and then
(iv) rinse the skin or hair.

41. The method of claim 40, wherein the instructions further comprise an initial usage step of wetting the personal cleansing composition prior to the lathering instruction (i).

42. The method of claim 40, wherein the instructions further comprise a final usage step of disposing any water-disintegratable substrate that remains after rinsing.

43. The method of claim 40, wherein the final usage step of disposing of any remaining substrate further comprises the usage step of disposing of the remaining substrate in a trash receptacle.

44. The method of claim 40, wherein the final usage step of disposing of any remaining substrate further comprises the disposing of the remaining substrate down a plumbing drain.
